SEQUENCE LISTING (51 sequences)

SEQ ID NO: 1 - RNA, 16 nt, artificial sequence
  flanking sequence miRNA
cuugggaauggcaagg

SEQ ID NO: 2 - RNA, 16 nt, artificial sequence
  flanking miRNA sequence
cucuugcuaucccaga

SEQ ID NO: 3 - DNA, 7762 bp, Homo sapiens
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcagtttcttattctccttcagattcctatctggtgtttccctgactttattcattcatcagtaaatatttactaaacatgtactatgtgcctggcactgttataggtgcagggctcagcagtgagcagacaaagctctgccctcgtgaagctttcattctaatgaaggacatagacagtaagcaagatagataagtaaaatatacagtacgttaatacgtggaggaacttcaaagcagggaaggggatagggaaatgtcagggttaatcgagtgttaacttatttttatttttaaaaaaattgttaagggctttccagcaaaacccagaaagcctgctagacaaattccaaaagagctgtagcactaagtgttgacatttttattttattttgttttgttttgttttttttgagacagttcttgctctatcagccaggctggagtgcactagtgtgatcttggctcactgcaacctctgcctcttgggttcaagtgattctcatgcctcagcctcctgtttagctgggattatagacatgcactgccatgcctgggtaattttttttttttcccccgagacggagtcttgctctgtcgcccaggctggagtgcagtggcgcgatctcagctcactgcaagctccgcttcccgagttcacgccattctcctgcctcagtctcccaagtagctgggactacaggcgcctgccaccacgtccagctaatttttttgtatttttaatagagacggggtttcaccgtgttagccaggatgatcttgatctcctgacctcgtcatccgccgaccttgtgatccgcccacctcggcctcccaaagtgctgggattacaggcatgagccactgtgcccggccacgcctgggtaatttttgtatttttagtagagatggggttttgccatgatgagcaggctggtctcgaactcccggcctcatgtgatctgcctgccttggcctcccaaagtgctaggattacaggcatgagccaccatacctggccagtgttgatattttaaatacggtgttcagggaaggtccactgagaagacagctttttttttttttttttttggggttggggggcaaggtcttgctctttaacccaggctggaatgcagtatcactatcgtagctcacttcagccttgaactcctgggctcaagtgatcctcccacctcaacctcacaatgtgttgggactataggtgtgagccatcacacctggccagatgatggcttttgagtaaagacctcaagcgagttaagagtctagtgtaagggtgtatgaagtagtggtattccagatggggggaacaggtccaaaatcttcctgtttcaggaatagcaaggatgtcattttagttgggtgaattgagtgagggggacatttgtagtaagaagtaaggtccaagaggtcaagggagtgccatatcagaccaatactacttgccttgtagatggaataaagatattggcatttatgtgagtgagatgggatgtcactggaggattagagcagaggagtagcatgatctgaatttcaatcttaagtgaactctggctgacaacagagtgaaggggaacaccggcaaaagcagaaaccagttaggaagccactgcagtgctcagataagcatggtgggttctgtcagggtaccggctgtcggctgtgggcagtgtgaggaatgactgactggattttgaatgcggaaccaactgcacttgttgaactctgctaagtataacaatttagcagtagcttgcgttatcaggtttgtattcagctgcaagtaacagaaaatcctgctgcaatagcttaaactggtaacaagcaagagcttatcagaagacaaaaataagtctggggaaattcaacaataagttaaggaacccaggctctttcttttttttttttttgaaacggagtttcgctcttgtcacccgggctggagtgcaatgatgtgatctcagctcactaaaacctctacctcctgggttcaagtgattcttctgcctcagcctcccaagtaactgggattacaggcgtataccaccatgcccagctaatttttgtgtttttagtagagatggggtttcaccatgttggccaggctggtctcgaacttctgacctcaggtgatccactcgcctcagcctgccaaagtgctgggattacaggtttgggccactgcacccggtcagaacccaggctctttcttatacttaccttgcaaacccttgttctcattttttccctttgtatttttattgttgaattgtaatagttctttatatattctggatactggattcttatcagatagatgatttgtaaaaactctcccttcctttggattgtctttttactttcttgatagtgtcttttgaagtgtaaaagtttttaattttgatgaagtcgagtttatctattttgtctttggttgctgtgcttcaagtgtcatatctaagaaatcattgtctaatccaaagtcaaaaaggtttactcctatgttttcttctaagaattttagagttttacatttaagtctgatccattttgagttaatttttatatatggttcaggtagaagtccaactttattcttttccatgtggttattcagttgtcccagcactgtttgttgaagagactattctttccccatggaattatcttagtacccttgttgaaaattaatcgtccttaattgtataaatttatttctagactgtcagttctacctgttggtctttatgtcgatcctgtgccagtaccatacagtcttgattactgaagtttgtgtcacagtttaaattcatgaaatgtgagttctccaactttgttccttttcaagattgatttggccatgctgggtcccttgcatttccgtacgaattgtaggatcagcttgtcagtttcaacaaagaagccaagtaggattctgagagggattgtgttgaatctgtagatcaacttggggagtattcgcatcttaacaatattgtcttccacctatgaacatgggcaaactttgtgtaaatggtcagattgtaagtatttcgggctgtgtgggcacagtgtctctgtcacagctacgcggctctgccattgtagcatgaaagtagccataagcaatatgtatgagtgtctgtgttccaatagaattttattaatgacaaggaagtttgaatttcatataattttcacctgtcatgagatagtatttgattattttggtcaaccatttaaaaatgtaaaaacatttcttagcttgtgaactagccaaaaatatgcaggttatagttttcccactcctaggttaaaatatgataggaccacatttggaaagcatttctttttttttttttttttttttttttgagacggagtttcactcttgttgcccaggctggagtgcagtggcgcgatctcggctcactgcaacctctgcctcccaggttcaagacattctcctgcacggcctccctagtagctgggattacaggcatgcgccaccacacccagctaattttgtatttttagtagagacggggtttctccatgttggtcaggctggtcttgaactcctgacctcaggtgatccacccgcctcagcctcccaaagtgctgggattacagggtgtgagccaccacaccctgctggaaagcatttcttttttggctgtttttgttttttttttaaactagttttgaaaattataaaagttacacatatacattataaaaatatcttcaagcagcacagatgaaaaacaaagcccttcttgcaagtctgtcatctttgtctaacttcctaagaacaaaagtgtttcttgtgtcttcttcccagattttaatatgcatatacaagcatttaaatgtgtcattttttgtttgcttgactgagatcacattacatatgtatttttttacttaacaatgtgtcatagatattgttccatagcagtacctgtaattcttattaattgctatgtaatattttagaatttctttttaaaagaggacttttggagatgtaaaggcaaaggtctcacatttttgtggctgtagaatgtgctggtgacatattctctctaccttgagaagtccccatccccatcacctccatttcctgtaaataagtcaaccacttgataaactacctttgaatggatccacactcaaaacatttagtcttattcagacaacaaggaggaaaaataaaataccttataaagcaaaaaaaa

SEQ ID NO: 4 - RNA, 196 nt, homo sapiens
gcugccgggacggguccaagauggacggccgcucagguucugcuuuuaccugcggcccagagccccauucauugccccggugcugagcggcgccgcgagucggcccgaggccuccggggacugccgugccgggcgggagaccgccauggcgacccuggaaaagcugaugaaggccuucgagucccucaaguccuuc

SEQ ID NO: 5 - RNA, 23 nt, homo sapiens
gagaccgccauggcgacccugga

SEQ ID NO: 6 - RNA, 23 nt, homo sapiens
agaccgccauggcgacccuggaa

SEQ ID NO: 7 - RNA, 24 nt, homo sapiens
gaugaaggccuucgagucccucaa

SEQ ID NO: 8 - RNA, 24 nt, homo sapiens
ggccuucgagucccucaaguccuu

SEQ ID NO: 9 - RNA, 22 nt, artificial sequence
  polynucleotide sequence targeting HTT mRNA
aaggacuugagggacucgaaga

SEQ ID NO: 10 - RNA, 21 nt, artificial sequence
  polynucleotide sequence targeting HTT mRNA
aaggacuugagggacucgaag

SEQ ID NO: 11 - protein, 88 aa, homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRP

SEQ ID NO: 12 - DNA, 119 bp, homo sapiens
acagcagcaaaagcagcaacagcagcagcagcagcagcagcagggggacctatcaggacagagttcacatccatgtgaaaggccagccaccagttcaggagcacttgggagtgatctag

SEQ ID NO: 13 - DNA, 95 bp, homo sapiens
gtgatgctatgagtgaagaagacatgcttcaggcagctgtgaccatgtctttagaaactgtcagaaatgatttgaaaacagaaggaaaaaaataa

SEQ ID NO: 14 - protein, 56 aa, homo sapiens
GDLSGQSSHPCERPATSSGALGSDLGDAMSEEDMLQAAVTMSLETVRNDLKTEGKK

SEQ ID NO: 15 - DNA, 190 bp, homo sapiens
gtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaa

SEQ ID NO: 16 - protein, 59 aa, homo sapiens
GDLSGQSSHPCERPATSSGALGSDLGKACSPFIMFATFTLYLTYELHVIFALHYSSFPL

SEQ ID NO: 17 - RNA, 22 nt, homo sapiens
aacacugguuuacaguuagaaa

SEQ ID NO: 18 - RNA, 22 nt, homo sapiens
aauuaggaaaacagugguuuaa

SEQ ID NO: 19 - RNA, 22 nt, homo sapiens
aaguaugcaagguaguuccaga

SEQ ID NO: 20 - RNA, 22 nt, homo sapiens
uacuucagaagagcuucggaag

SEQ ID NO: 21 - RNA, 22 nt, homo sapiens
gagacgagaagccuacuuugaa

SEQ ID NO: 22 - RNA, 22 nt, artificial sequence
  polynucleotide sequence targeting ATXN3 mRNA
uuucuaacuguaaaccaguguu

SEQ ID NO: 23 - RNA, 22 nt, artificial sequence
  polynucleotide sequence targeting ATXN3 mRNA
uuaaaccacuguuuuccuaauu

SEQ ID NO: 24 - RNA, 22 nt, artificial sequence
  polynucleotide sequence targeting ATXN3 mRNA
ucuggaacuaccuugcauacuu

SEQ ID NO: 25 - RNA, 22 nt, artificial sequence
  polynucleotide sequence targeting ATXN3 mRNA
cuuccgaagcucuucugaagua

SEQ ID NO: 26 - RNA, 22 nt, artificial sequence
  polynucleotide sequence targeting ATXN3 mRNA
uucaaaguaggcuucucgucuc

SEQ ID NO: 27 - DNA, 17 bp, artificial sequence
  primer -19 fw
aggaaccgctgcaccga

SEQ ID NO: 28 - DNA, 27 bp, artificial sequence
  primer 431 rv
gagacctcctaaaagcattatgtcatc

SEQ ID NO: 29 - DNA, 22 bp, artificial sequence
  primer ex2 fw
aagaaggaactctcagccacca

SEQ ID NO: 30 - DNA, 23 bp, artificial sequence
  primer ex2 rv
ctgagagactgtgccacaatgtt

SEQ ID NO: 31 - DNA, 23 bp, artificial sequence
  primer 347 fw
tcctcatcaggcctaagagctgg

SEQ ID NO: 32 - DNA, 22 bp, artificial sequence
  primer 785 rv
tgaaaactgagcaccaccaatg

SEQ ID NO: 33 - DNA, 18 bp, artificial sequence
  primer 5′UTR fw
cttggttccgcttctgcc

SEQ ID NO: 34 - DNA, 20 bp, artificial sequence
  primer 5′UTR rv
tggagcctactggcactacg

SEQ ID NO: 35 - DNA, 24 bp, artificial sequence
  5′UTR p
cagagccccattcattgccttgct

SEQ ID NO: 36 - DNA, 17 bp, artificial sequence
  primer 135 fw
cttgcggggtctctggc

SEQ ID NO: 37 - DNA, 18 bp, artificial sequence
  primer 200 rv
tcagcgagtccctggctg

SEQ ID NO: 38 - DNA, 25 bp, artificial sequence
  155 p
cctcagaggagacagagccgggtca

SEQ ID NO: 39 - DNA, 27 bp, artificial sequence
  primer 431 rv
gagacctcctaaaagcattatgtcatc

SEQ ID NO: 40 - DNA, 25 bp, artificial sequence
  371 p
agtgcaggacagcgtgagagatgtg

SEQ ID NO: 41 - DNA, 37 bp, artificial sequence
  Ex2 p
agaaagaccgtgtgaatcattgtctaacaatatgtga

SEQ ID NO: 42 - DNA, 52 bp, artificial sequence
  QT 3′RACE
ccagtgagcagagtgacgaggactcgagctcaagcttttttttttttttttt

SEQ ID NO: 43 - DNA, 18 bp, artificial sequence
  Q0
ccagtgagcagagtgacg

SEQ ID NO: 44 - DNA, 18 bp, artificial sequence
  Qi
gaggactcgagctcaagc

SEQ ID NO: 45 - DNA, 24 bp, artificial sequence
  primer 571 fw
aaccaggttttaagcatagccaga

SEQ ID NO: 46 - DNA, 30 bp, artificial sequence
  primer 622 fw
agttggatgagttgtatttgtcaagtacat

SEQ ID NO: 47 - DNA, 513 bp, Mus musculus
  misc_feature (4)..(6): n is a, c, g, or t
  misc_feature (297)..(297): n is a, c, g, or t
  misc_feature (383)..(383): n is a, c, g, or t
  misc_feature (509)..(509): n is a, c, g, or t
cgtnnnatttcttaggtgtgattattaataaaaaactatatgtgtgcatatatatgaaagagtcgacttatacttaactgcctatcgattttttgttctatataaaacggatacattggtggtgctcagttttcaccggggaatgaattttactagtgttgcagacaggcttgttttagaacataggccactctgactctgactttgtgccagtaaaagttcctgtttagttctttgctgacatcttatagatctttggaagctagctgcttgtgactggagagaatattgaaacanaagagagaccatgagtcacagtgctctaagagaaaagagacgctcaaaacatttcctggaaatccatgctgagtgttgagccctgngctctcttgcagctcagtcctttctctcaactctgggcattttatttctaatctggatttgtataattaataaggagaacttttgggaacaacctactaaagaatgtcatcattaaaactcattanaatc

SEQ ID NO: 48 - DNA, 79 bp, mus musculus
  misc_feature (5)..(5): n is a, c, g, or t
  misc_feature (7)..(7): n is a, c, g, or t
  misc_feature (9)..(9): n is a, c, g, or t
  misc_feature (15)..(15): n is a, c, g, or t
ggtgnantntattangtgtgattattataaaaaactatatgtgtgcatataaaaaaaaaaaaaaaaaaaaaaaaaaaaa

SEQ ID NO: 49 - DNA, 319 bp, mus musculus
  misc_feature (4)..(4): n is a, c, g, or t
  misc_feature (8)..(8): n is a, c, g, or t
gccncccngtgagcaggctttccggcccgggccctcgtcttgcggggtctctggcctccctcagaggagacagagccgggtcaggccagccagggactcgctgaggggcgtcacgactccagtgccttcgccgttcccagtttgcgaagttagggaacgaacttgtttctctcttctggagaaactggggcggtggcgcacatgactgttgtgaagagaacttggagaggcagagatctctagggttacctcctcatcaggcctaagagctgggagtgcaggacagcgtgagagatgtgcgggtagtggatgacataat

SEQ ID NO: 50 - DNA, 22 bp, artificial sequence
  DNA sequence corresponding with sequence targeting HTT mRNA
aaggacttgagggactcgaaga

SEQ ID NO: 51 - protein, 50 aa, Homo Sapiens
PPPPPPPPPPPQLPQPPPQAQPLLPQPQPPPPPPPPPPGPAVAEEPLHRP

(19) United States
(12) Patent Application Publication
(10) Pub. No.: US 2022/0213482 A1
(43) Pub. Date: Jul. 7, 2022

(54) TARGETING MISSPLICED TRANSCRIPTS IN GENETIC DISORDERS

(71) Applicant: Uniqure IP B.V., Amsterdam (NL)

(72) Inventors: Sander Jan Hendrick VAN DEVENTER, Amsterdam (NL); Melvin Maurice EVERS, Amsterdam (NL); Maria SOGORB-GONZÁLEZ, Amsterdam (NL); Pavlina Stefanova KONSTANTINOVA, Amsterdam (NL); Astrid VALLES-SANCHEZ, Amsterdam (NL)

(21) Appl. No.: 17/695,289

(22) Filed: Mar. 15, 2022

Related U.S. Application Data
(63) Continuation of application No. PCT/EP2020/075871, filed on Sep. 16, 2020.

(30) Foreign Application Priority Data
Sep. 16, 2019 (EP) 19197533.3

(51) Int. Cl. C12N 15/113; C12N 15/86

(52) U.S. Cl. CPC C12N 15/113; C12N 2750/14143; C12N 2310/14; C12N 15/86

(57) ABSTRACT

The present invention relates to repeat expansion disorders. Missplicing is understood to be a general phenomenon that can occur in repeat expansion disorders wherein the DNA and/or RNA sequence of repeat sequences in expanded repeat disorders can cause such aberrant transcription and/or aberrant splicing, resulting in misspliced transcripts, i.e. transcripts that do not have the putative splicing as observed e.g. for corresponding non-diseased genes. Such misspliced transcripts can be in particular associated with disease. Hence, the current invention now provides means and methods for targeting misspliced transcripts which is highly useful for the treatment of expanded repeat disorders.

### CROSS-REFERENCE TO RELATED APPLICATIONS

The present application is a Continuation of International Patent Application No. PCT/EP2020/075871, filed Sep. 16, 2020, which claims priority to European Patent Application No. 19197533.3 filed Sep. 16, 2019; the entire contents of all of which are hereby incorporated by reference.

### SEQUENCE LISTING

The instant application contains a Sequence Listing which has been submitted electronically in ASCII format and is hereby incorporated by reference in its entirety. Said ASCII copy, created on Mar. 14, 2022, is named Sequence_Listing.txt and is 23,547 bytes in size.

### INTRODUCTION

Pharmaceutical interventions aimed at reducing expression of a gene associated with a disease have since long been the subject of pharmaceutical development of polynucleotides. Strategies for reduction of transcripts expressed from a gene that may be utilized include sequence-specific targeting of RNA transcripts using approaches such as antisense technology, e.g. ASO's (antisense oligonucleotides), and RNAi. In recent years, approval has been obtained for such products. For example, patisiran (Alnylam) is a small interfering RNA drug, aimed at sequence-specifically targeting an abnormal form of transthyretin, and volanesorsen (Ions) is an antisense therapeutic oligonucleotide (ASO) that targets the messenger RNA for apolipoprotein C3 (apo-CIII) for the treatment of hypertriglycidemia, familial chylomicronemia syndrome and familial partial lipodystrophy. In these treatments, the polynucleotides have to be administered repeatedly as these drugs are cleared from the bodily system. Further treatments that are currently under development are gene therapy approaches wherein therapeutic polynucleotides are expressed in the treated individual in vivo, providing a continuous supply thereof without requiring repeated administration.

Repeat expansion disorders are genetic disorders caused by an expansion of a repeat sequence which exceeds the normal, stable threshold. Expanded trinucleotide repeat disorders were discovered first. Recently tetra-, penta-, hexa- and even dodeca-nucleotide repeat expansions have been identified to cause human disease. The main category of repeat expansion disorders is of trinucleotide CAG repeats, which are found i.a. in Huntington disease and in spinocerebellar ataxia (SCA) wherein said CAG repeats occur in protein coding portions of the affected gene. In the Huntingtin gene, also called the HTT or HD (Huntington disease) gene, the number of CAG repeats in healthy individuals is in the range of 6 to 35, whereas patients diagnosed as having Huntington disease have more than 36 CAG repeats. Patients with 36 and 39 repeats in the HTT gene are in the ‘reduced penetrance’ range, which means that some people in this range will develop HD symptoms, while others will not. Similarly, in SCA3 the number of CAG repeats in healthy individuals is in the range of 12-40, whereas the number of repeats observed in SCA3 patients can exceed 55 CAG repeats. In the intermediate range for SCA3, not all individuals may present with SCA3 disease symptoms. As the trinucleotide CAG, when in-frame, represents a codon for a glutamine amino acid residue (Gln or Q), such trinucleotide CAG repeat disorders, which occur in over half of known trinucleotide repeat disorders, are also referred to as polyQ or polyglutamine diseases. Proteins or protein fragments of Huntingtin protein or the like containing expanded repeat encoded amino acid sequences, e.g. an expanded polyglutamine repeat, are found to be toxic and are also found to aggregate and accumulate in cells. Likewise, RNA foci containing repeat expansion sequences can be observed in expanded repeat disorders that may also contribute to disease.

Repeat expansion disorders share mostly a striking genotype-phenotype correlation between repeat expansion length and disease severity. The longer the repeat expansion, the more severe the disease and the earlier the onset of disease. Most repeat expansion disorders primarily involve a toxic gain of function. Targeting the expanded repeat sequences is a challenge, mainly because expanded repeat sequences are highly structured, rendering these sequences less accessible and also because such repeat sequences may occur in many genes, some of which are important for cellular functioning, rendering selectivity when targeting such sequences difficult. Strategies under development for the treatment of repeat expansion disorders or the like have focused on targeting disease associated transcripts, e.g. via (i) selectively targeting mutant (expanded repeat containing) transcripts (utilizing single nucleotide polymorphisms associated with disease by specifically targeting a single nucleotide that is in linkage disequilibrium with the expanded CAG repeat), so called allele-specific targeting or via (ii) specifically targeting both wild type and mutant (expanded repeat containing) transcripts, so called total targeting. Such approaches are mainly based on the paradigm that targeting any exon, 5′ or 3′ UTR sequence of an RNA transcript is to effectively reduce production of toxic RNAs and/or toxic proteins, and/or fragments thereof.

### SUMMARY OF THE INVENTION

It has now been observed that in a repeat expansion disease disorder, i.e. Huntington disease, aberrant splicing occurs, i.e. the splice donor site adjacent to the exon containing the expanded repeat (exon1) of transcripts produced is not used (Neueder et al. 2017, Scientific Reports May 2; 7: 1307; and as depicted in FIGS. 1 and 2). This results in a misspliced truncated transcript containing exon1 comprising the trinucleotide CAG repeat, and a portion of intron1 and a subsequent polyA tail (because of the presence of a cryptic poly A signal in HTT intron 1). When translated, a truncated HTT protein is produced, also referred to as exon1 HTT protein, comprising the expanded polyQ sequence which is associated with disease. Such missplicing has been observed in human patients and also in Huntington disease mouse models. Similarly, in a SCA3 mouse model, such missplicing has also been observed, and has been implicated to result in accelerated toxic protein aggregation (Human Molecular Genetics, 2015, Vol. 24 No. 5 pp. 1211-24 and FIG. 10).

Hence, missplicing in accordance with the invention is understood to be a general phenomenon that can occur in repeat expansion disorders. mRNA splicing occurs in the cell nucleus concurrently with transcription and polyadenylation. During this process, splicing factors join exons and exclude introns. The DNA and/or RNA sequence of repeat sequences in expanded repeat disorders in accordance with the invention can cause such aberrant transcription and/or aberrant splicing, resulting in misspliced transcripts, i.e. transcripts that do not have the putative splicing as observed for non-diseased genes. Hence, the current inventors now adopted the approach of targeting misspliced transcripts. The current inventors have deviated from the conventional approach relying on the paradigm that targeting any exon, 5′ or 3′ UTR sequence of an RNA transcript is to effectively reduce production of toxic proteins and/or toxic RNAs in repeat expansion disorders. Instead, the current inventors now advantageously focus on a reduction of both full length and misspliced transcripts. For example, one can sequence-specifically target sequences of misspliced transcripts. The inventors now show here that by following that approach, a strong reduction of misspliced transcripts containing the repeat sequence can be achieved, which is highly beneficial as these particular misspliced transcripts are to contribute significantly to disease. Misspliced transcripts can be toxic and the proteins encoded by said misspliced transcripts can be toxic as well. The approach taken by the inventors has the additional advantage that, apart from a reduction of misspliced transcripts, also a reduction of the full mutant HTT is achieved. The current invention here now provides for methods and means for advantageously targeting such misspliced transcripts for the treatment of repeat expansion disorders.

### FIGURES

FIG. 1. This figure provides, for Huntington Disease, an overview about aberrant/missplicing in expanded repeats. HTT is alternatively spliced into a truncated isoform in HD cells. In HD fibroblasts and brain tissue, the splicing factor SRSF6 processes mutant HTT mRNA into a third alternatively polyadenylated splice isoform that terminates in intron 1. This isoform may be translated into the pathogenic N-terminal HTT protein prone to aggregation and toxicity. Taken from: J Huntingtons Dis. 2018; 7(2): 101-108.

FIG. 2. Schematic representation of the generation of the pathogenic HTT protein by aberrant splicing. The huntingtin gene is transcribed into mRNA and spliced, generating a full-length protein. However, in HD patients a short mRNA transcript containing exon1-Intron1 sequence (Exon1 HTT) is also generated due to aberrant splicing. This results in the translation of a highly pathogenic HTT protein, which form aggregates and induce toxicity in neuronal cells.

FIGS. 3A and 3B: HD mouse models:
FIG. 3A: Schematic of the HTT gene in WT and Q175 KI HD mouse models. Both Q175 KI models carry the human Exon 1 sequence inserted in HTT mouse gene with expansion of 175 CAG repeats.
FIG. 3B: Summary of animal groups and brain areas used in this study.

FIG. 4: Schematic of mouse HTT gene and location of primers used in this study.

FIGS. 5A, 5B and 5C: Detection of Exon 1 HTT mRNA in Q175 KI mice models:
FIG. 5A: Scheme of the HTT polyadenylated mRNAs reverse transcribed with oligodT primer. The arrows show the binding location of forward and reverse primers.
FIG. 5B and FIG. 5C. 3′RACE results showed the presence of two Intron1-containing polyadenylated mRNA products in Q175 HET KI (B) and in Q175 HOM KI (C) mice. Intron1-containing polyadenylated transcripts represent mis-spliced Exon1 HTT mRNA and were not detected in WT mice.

FIG. 6: RT-PCR analysis to confirm the presence of full-length HTT mRNA (Exon1-2) and misspliced Exon1 HTT mRNA (Exon1-Intron1) in WT and Q175 KI HET mice.

FIG. 7: Quantification of relative expression of the full-length HTT mRNA (Exon1-2 and exon 2) and the mis-spliced HTT mRNA (Early intron1 and Intron1) by TaqMan RT-qPCR in WT, Q175 KI HET and HOM mouse models.

FIGS. 8A and 8B: miHTT expression levels in striatum (FIG. 8A) and cortex (FIG. 8B) after treatment with low dose and high dose of AAV-miHTT in Q175 KI HOM

FIGS. 9A, 9B, 9C, 9D and 9E: Lowering of full-length and Exon1 HTT mRNA by AAV-miHTT in Q175 KI HOM mice:
FIG. 9A: Schematic of mouse gene and primer sets used to quantify HTT mRNA expression.
FIG. 9B and 9C: Expression levels in the striatum region of full-length (FIG. 9B) and Exon 1 (FIG. 9C) HTT mRNA upon treatment with low and high dose of AAV-miHTT.
FIG. 9D and 9E: Expression levels in the cortex region of full-length (FIG. 9D) and Exon1 (FIG. 9E) HTT mRNA upon treatment with low and high dose of AAV-miHTT.

FIG. 10. Missplicing of the mutant ATXN3 transcript potentially accelerates ataxin-3 aggregation. Diagram of 3′ missplicing of the human ATXN3 transcript in SCA3 knock-in mice, showing the Intron 10-containing ATXN3 transcript (right) generated from retention of intron 10 due to missplicing, which encodes a hydrophobic segment that may accelerate mutant ATXN3 aggregation. Taken from: Hum Mol Genet. 2015 Mar. 1; 24(5): 1211-1224.

FIGS. 11A and 11B. Detection of exon 1 mRNA transcript in Q175KI HET mice. Quantification of relative expression of (FIG. 11A) the full-length HTT mRNA (5′UTR, Exon1-2 and exon 64-65) and (FIG. 11B) the mis-spliced HTT mRNA (Early intron1, Intron1, human exon1-intron1) by TaqMan RT-qPCR in WT and Q175KI HET mice. Relative expression is calculated based of the geometric mean of three housekeeping genes (GAPDH, PGK1 and HPRT). Bar graphs represent mean+SEM.

FIGS. 12A and 12B. miHTT expression levels in left frontal cortex (FIG. 12A) and left caudal cortex (FIG. 12B) after 2 months treatment with low dose and high dose of AAV-miHTT in Q175KI HET mice. Graphs represent mean+SEM.

FIGS. 13A, 13B and 13C. Lowering of cortical full-length and Exon1 HTT mRNA in Q175 KI HET mice 2 months after striatal administration of AAV5-miHTT:
FIG. 13A: Schematic of mouse gene and primer sets used to quantify HTT mRNA expression.
FIGS. 13B and 13C: Expression levels in the left frontal cortex relative to vehicle-treated mice of full-length HTT mRNA (FIG. 13B) and Exon 1 HTT mRNA (FIG. 13C) upon treatment with low and high dose of AAV-miHTT. Statistical analysis was performed by one-way ANOVA and Dunnett's multiple comparisons tests. *p<0.05, ****p<0.001.

FIGS. 14A and 14B: Correlation analysis between miRNA biodistribution and HTT mRNA expression in the frontal cortex;
FIG. 14A: correlation between miRNA expression levels (molecules/cell) and relative expression of exon 64-65 mRNA (representing full-length HTT mRNA).
FIG. 14B: Correlation between miRNA expression levels (molecules/cell) and relative expression of human exon1-intron 1 mRNA (representing mutant exon 1 mRNA). Statistical analysis was performed by non-parametric Spearmen test (r) and p<0.05.

FIGS. 15A and 15B: HD mouse models:
FIG. 15A: Schematic of the HTT gene in WT, Q175 KI HD mouse models, and Q175KI HOM model. Both Q175 KI models carry the human Exon 1 sequence inserted in HTT mouse gene with expansion of 175 CAG repeats.
FIG. 15B. Summary of animal groups and brain areas used in this study, which were used in the same protocol for the study of FIG. 3, as described below.

### EMBODIMENTS

In one embodiment, a polynucleotide for use in the treatment of a repeat expansion disorder is provided, wherein said repeat expansion disorder results in missplicing 3′ from said repeat expansion, producing a misspliced transcript, and wherein said polynucleotide is capable of inducing a reduction of said misspliced transcript.

As said, repeat expansion disorders are genetic disorders caused by an expansion of a repeat sequence in a gene (hereinafter also called the “diseased-gene”) that can cause transcription, splicing and/or polyadenylation to be different from a corresponding gene that lacks the expanded repeat sequence (hereinafter also called the “non-diseased-gene”), producing alternatively organized transcripts as compared with transcripts from corresponding genes not associated with disease. Missplicing (or aberrant splicing), at least in the context of expansion repeat disorders such as Huntington Disease or SCA3, is understood to mean that one or more sequences that function in splicing (such as splice donor and/or acceptor sequences) as observed for non-diseased genes are not utilized in the process of gene expression from the diseased gene. Without being bound by theory, the structure of the expanded repeat sequence is underlying such missplicing events. It is understood that missplicing can be a chance event, i.e. not all transcripts are misspliced. The longer the expanded repeat sequence, the more missplicing events may occur, hence, explaining in part an association of expanded repeat sequence length with disease severity. The expanded repeat sequences within the DNA and/or RNA are enabling for aberrant transcription, splicing and/or polyadenylation, thereby producing such aberrant RNA transcripts. Such aberrant transcription, splicing and/or polyadenylation (or missplicing) typically occurs downstream (i.e. 3′) from the expanded repeat sequence. Hence, instead of referring to misspliced transcripts, one may also refer to aberrant transcripts. Transcripts from expanded repeat sequences 5′ from the expanded repeat sequence will be organized like in transcripts produced from genes not associated with disease, whereas 3′ from the expanded repeats sequence the transcript will be aberrantly organized. Whichever terminology used, i.e. referring to an aberrant transcript or a misspliced transcript, the current invention now provides for polynucleotides selected for being capable of reducing such misspliced transcripts.

Reducing misspliced transcripts with a polynucleotide in accordance with the invention provides benefit to patients suffering from an expanded repeat disorder, as such misspliced transcripts are associated with disease. Hence, targeting misspliced transcripts in expanded repeats disorders is highly useful in the treatment of such disorders. Preferably such treatments are of human subjects identified as carrying a gene having an expanded repeat associated with disease. It is understood that such a treatment may be a prophylactic treatment, i.e. subjects, which are preferably human subjects, are treated prior to observing any symptoms of disease. Such subjects can be identified early on, e.g. identified through genetic screening at birth, or because subjects are suspected of having inherited the disease because family members have been diagnosed as having such a disorder. Alternatively, treatment can commence when subjects, preferably human subjects, are diagnosed with the disease after disease symptoms have manifested.

A reduction of misspliced transcripts can easily be determined by e.g. determining the amount of misspliced transcripts, e.g. in an in vitro assay in patient derived cells e.g. taken at various times prior, during and/or after treatment, or by use of the polynucleotides in an appropriate animal model, such as described in the example section. The amount of misspliced transcript can be determined e.g. by designing primers that can selectively amplify misspliced transcripts by selecting primer binding sites that are unique to a misspliced transcript. For example, misspliced transcripts can be detected from isolated cytoplasmic material or from a whole cell lysate. Alternatively, as misspliced transcripts may produce aberrant proteins, which proteins can comprise a different amino acid sequence as compared with a wild-type protein or which proteins are abundantly present as compared to wildtype, especially as observed for longer expanded repeats, the detection of such aberrant proteins and reduction thereof, is representative of misspliced transcripts and a reduction thereof. Such a protein may be detected by using antibodies specific thereto. A reduction of misspliced transcripts (or its representative aberrant protein) may be detected in appropriate animal models and in in vitro assays wherein the amount of misspliced transcripts prior to treatment is taken as reference value. A reduction may also be detected in patient derived samples, e.g. in patients undergoing treatment or in patient samples tested with regard to suitability of the treatment in accordance with the invention. For example, in a neurodegenerative disease caused by a repeat expansion, a reduction of aberrant proteins encoded by misspliced transcripts may be detected in a CSF sample. In any case, whether or not a polynucleotide in accordance with the invention is capable of reducing misspliced transcripts can be well determined, either in an appropriate model or from patient samples before and/or in treatment in accordance with the invention.

In another embodiment, a polynucleotide for use in the treatment of a repeat expansion disorder is provided, wherein said repeat expansion is a CAG repeat, wherein said repeat expansion disorder results in missplicing 3′ from said repeat expansion, producing a misspliced transcript, and wherein said polynucleotide is capable of inducing a reduction of said misspliced transcript. Preferably, such a CAG repeat is comprised in an exon. More preferably, said polynucleotide provided for use in the treatment of a repeat expansion disorder in accordance with the invention, comprises a use wherein said misspliced transcripts contain an exon comprising the CAG repeat and containing an intron sequence which is 3′ and adjacent from said exon with the CAG repeat. Most preferably, said CAG repeat is in-frame with the reading frame of the encoded protein. As described above, repeat expansions, such as a CAG repeat, when e.g. contained in an exon sequence, can cause missplicing, i.e. cause splice donor site to not be utilized. For example, when a CAG repeat is comprised in exon1 (as is the case in Huntington's disease), the splice donor of a subsequent intron1 sequence may not be used. When this occurs, the result is a transcript that has an exon1 sequence followed by the intron1 sequence adjacent to the intron1 sequence. Such misspliced transcripts can be found in the cytoplasm. Hence, in the cytoplasm, misspliced transcripts contain sequences normally not found in non-aberrant transcripts.

In a further embodiment, a polynucleotide for use in accordance with the invention is provided, being for use in the treatment of a CAG repeat expansion disorder, and wherein said misspliced transcript that is reduced comprises an exon comprising the CAG repeat and containing an intron sequence which is 3′ and adjacent from said exon with the CAG repeat, said misspliced transcripts further comprise a polyA 3′ adjacent to said intron sequence. As described above, it is understood that because of a repeat expansion, transcription, splicing and/or polyadenylation may be affected due to an expanded repeat sequence. In particular, it has been observed in CAG repeat expansion disorders that polyadenylation may be affected, i.e. cryptic polyadenylation (polyA) sequences that may be present in an intronic sequence, may be used. It is understood that cryptic polyadenylation signals are sequences that are normally not used for polyadenylation. This is because these are often present in an intron, and the splicing mechanism normally suppresses polyadenylation from these cryptic polyadenylation signals. Because of the presence of an expanded repeat sequence, the splicing can be suppressed, mediated by abnormal binding of splicing factors (like SRSF6) to the CAG repeat. This can subsequently interfere with the formation of the spliceosome or expose cryptic polyadenylation sites. Splicing factors thus regulate splicing and facilitate translation of partially spliced transcripts. Hence, in such a scenario, the exon sequence is transcribed comprising the CAG repeat, followed by the subsequent intronic sequence, which subsequently is polyadenylated at the cryptic polyA sequence present in the subsequent intronic sequence. The transcript thus comprising subsequently from 5′ to 3′, an exon sequence with an expanded repeat sequence, such as a CAG repeat sequence, followed by an intron sequence until the polyadenylation signal, followed by a polyA tail.

The misspliced transcript that is produced can produce a protein and will have a protein amino acid sequence that is coded by an exon, which can continue in the reading frame corresponding with a subsequent intron sequence. Protein translation can terminate when an in-frame stop-codon is reached e.g. within a subsequent intron sequence. As translation from transcripts occurs in the cytoplasm, the polynucleotide for use in the treatment of a repeat expansion disorder in accordance with the invention is to provide for a reduction of said misspliced transcripts in the cytoplasm. Said reduction can be achieved by reducing said misspliced transcripts in the cytoplasm. Said reduction can be achieved by reducing said misspliced transcripts in the nucleus, before they are exported to the cytoplasm, also resulting in a reduction of misspliced transcripts in the cytoplasm. Said reduction can also be achieved by reducing said misspliced transcripts in cytoplasm and in the nucleus. Whichever means of reduction is used, the result is a reduction of misspliced transcripts in the cytoplasm, resulting in e.g. a reduction of RNA foci and/or protein aggregates produced therefrom in the cytoplasm.

Said means of reduction of misspliced transcripts preferably comprises the use of a polynucleotide that is complementary to said misspliced transcript. With regard to complementarity of a polynucleotide to the misspliced transcripts it is understood that complementarity means that nucleotides of the polynucleotide form base pairs with a target sequence comprised within said misspliced transcript. Hence, a polynucleotide is designed such that it targets a sequence within said misspliced transcript. In this context, reference can also be made to sequence-specifically targeting misspliced transcripts.

For example, in case the polynucleotide comprises RNA nucleotides, the nucleotides cytosine and guanine (C and G) can form a base pair, guanine and uracil (G and U), and uracil and adenine (U and A). The complementarity can be over the entire length of the polynucleotide, which means that all nucleotides within the polynucleotide can base pair with the target sequences (also referred to as full complementarity). The complementarity can also be substantial, i.e. it may not be required to have the polynucleotide and target sequence to be fully complementary. In a further embodiment, the complementarity between the polynucleotide and the target sequence consists of having no mismatches, one mismatched nucleotide, or two mismatched nucleotides. It is understood that one mismatched nucleotide means that over the entire length of the polynucleotide that base pairs with the target sequence one nucleotide does not base pair with the target nucleotide. The length of the target nucleotide comprised within the misspliced transcript may be in the range of 13-25 nucleotides. Accordingly, the length of the polynucleotide in accordance with the invention may have the same length as the target nucleotide.

In a further embodiment, the polynucleotide for use in the treatment of a repeat expansion disorder in accordance with the invention is complementary to said misspliced transcripts and said complementarity is 5′ from the repeat expansion. Targeting a sequence 5′ from the expanded repeat sequence may ensure that whatever missplicing occurs downstream from the expanded repeat sequence, the misspliced transcripts produced are efficiently reduced. Concomitantly, any transcripts which have not underwent missplicing may be reduced as well. Hence, targeting a sequence 5′ from expanded repeat sequences may have the benefit that both misspliced and regularly spliced transcript containing expanded repeat sequences can be reduced, of which both can be associated with disease. Polynucleotides designed to target misspliced transcripts are described in the example section (including double stranded RNAs inducing RNA interference and antisense oligonucleotides).

In a further embodiment, the polynucleotide in accordance with the invention may be an antisense oligonucleotide or comprised in a double stranded RNA capable of inducing RNA interference.

In one embodiment, the polynucleotide in accordance with the invention is an antisense oligonucleotide. Antisense oligonucleotides are well known in the art (e.g. inotersen and volanesorsen (Ions) are antisense oligonucleotides that have been approved for human use), likewise, target sequences can be selected and polynucleotides designed in accordance with the invention to target misspliced transcripts. Such antisense oligonucleotides can include RNA and/or DNA nucleotides. Such antisense nucleotides can include synthetic nucleotides. Such polynucleotides may have modifications that provide stability to the polynucleotide (e.g. extend half-life), can increase affinity to its target sequence and/or enhance delivery.

In one embodiment, the polynucleotide in accordance with the invention is comprised in a double stranded RNA capable of inducing RNA interference. Double stranded RNA capable of inducing RNA interference can also be utilized and a polynucleotide in accordance with the invention can be designed to target misspliced transcripts. RNA interference may be preferred as it can easily be employed using a gene therapy approach that can provide for a durable reduction of misspliced transcripts.

As said, a double stranded RNA can be provided capable of inducing RNA interference (RNAi) and comprising the polynucleotide that targets misspliced transcripts, resulting in a reduction thereof. Double stranded RNA structures that are suitable for inducing RNAi are well known in the art. For example, a small interfering RNA (siRNA) comprises two separate RNA strands, one strand comprising a first RNA sequence and the other strand comprising a second RNA sequence. The first RNA sequence representing a polynucleotide in accordance with the invention that is to target misspliced transcripts. The first RNA sequence, i.e. a polynucleotide in accordance with the invention, can be comprised in the guide strand of the double stranded RNA, also referred to as antisense strand as it is complementary (“anti”) to the sense target sequence, i.e. to a sequence comprised in a misspliced transcript. The second RNA sequence is comprised in the passenger strand, also referred to as “sense strand” as it may have substantial sequence identity with or be identical with the target sequence. The first and second RNA sequences are comprised in a double stranded RNA and are substantially complementary. The said double stranded RNA according to the invention is to induce RNA interference to thereby reduce misspliced transcript expression. Hence, it is understood that substantially complementary means that it is not required to have all the nucleotides of the first and second RNA sequences base paired, i.e. to be fully complementary. As long as the double stranded RNA is capable of inducing RNA interference to thereby sequence-specifically target a sequence comprised in misspliced transcripts, such substantial complementarity is contemplated in the invention.

An siRNA design that is often used typically involves 19 consecutive base pairs with 3′ two-nucleotide (2 nt) overhangs. This design is based on observed Dicer processing of larger double stranded RNAs that results in siRNAs having these features. The 3′-overhang may be comprised in the first RNA sequence. The 3′-overhang may be in addition to the first RNA sequence. The length of the two strands of which an siRNA is composed may be 19, 20, 21, 22, 23, 24, 25, 26 or 27 nucleotides or more. Each of the two strands comprises the first and second RNA sequence. The strand comprising the first RNA sequence may also consist thereof. The strand comprising the first RNA sequence may also consist of the first RNA sequence and the overhang sequence.

siRNAs may also serve as Dicer substrates. For example, a Dicer substrate may be a 27-mer consisting of two strands of RNA that have 27 consecutive base pairs. The first RNA sequence is positioned at the 3′-end of the 27-mer duplex. At the 3′-end, like with siRNAs, is a two-nucleotide overhang. The 3′-overhang may be comprised in the first RNA sequence. The 3′-overhang may be in addition to the first RNA sequence. 5′ from the first RNA sequence, additional sequences may be included that are either complementary to the sequence adjacent to the target sequence or not. The other end of the siRNA Dicer substrate is blunt ended. This Dicer substrate design results in a preference in processing by Dicer such that an siRNA is formed like the siRNA design as described above, having 19 consecutive base pairs and 2 nucleotide overhangs at both 3′-ends. In any case, siRNAs, or the like, are composed of two separate RNA strands (Fire et al. 1998, Nature. 1998 Feb. 19; 391 (6669):806-811) each RNA strand comprising or consisting of the first and second RNA sequence, the first RNA sequence representing a polynucleotide for use in the treatment of a repeat disorder in accordance with the invention.

The double stranded RNA according to the invention does not require both first and second RNA sequences to be comprised in two separate strands. The first and second RNA sequences can also be comprised in a single polynucleotide, a single strand of RNA, such as e.g. an shRNA. A shRNA may comprise from 5′-second RNA sequence-loop sequence-first RNA sequence-optional 2 nt overhang sequence-3′. Alternatively, a shRNA may comprise from 5′-first RNA sequence-loop sequence-second RNA sequence-optional 2 nt overhang sequence-3′. Such an RNA molecule forms intramolecular base pairs via the substantially complementary first and second RNA sequence. Suitable loop sequences are well known in the art (i.a. as shown in Dallas et al. 2012 Nucleic Acids Res. 2012 October; 40(18):9255-71 and Schopman et al., Antiviral Res. 2010 May; 86(2):204-211).

The loop sequence may also be a stem-loop sequence, whereby the double stranded region of the shRNA is extended. Without being bound by theory, like the siRNA Dicer substrate as described above, an shRNA is usually processed by Dicer to obtain e.g. an siRNA having an siRNA design such as described above, having e.g. 19 consecutive base pairs and 2 nucleotide overhangs at both 3′-ends. In case the double stranded RNA is to be processed by Dicer, it is preferred to have the first and second RNA sequence at the end. A double stranded RNA according to the invention may also be incorporated in a pre-miRNA or pri-mi-RNA scaffold. Micro RNAs, i.e. miRNA, are guide strands that originate from double stranded RNA molecules that are expressed e.g. in mammalian cells. A miRNA is processed from a pre-miRNA precursor molecule, similar to the processing of an shRNA or an extended siRNA as described above, by the RNAi machinery and incorporated in an activated RNA-induced silencing complex (RISC) (Tijsterman M, Plasterk RH. Dicers at RISC; the mechanism of RNAi. Cell. 2004 Apr. 2; 1 17(1):1 -3).

Without being bound by theory, a pre-miRNA is a hairpin molecule that can be part of a larger RNA molecule (pri-miRNA), e.g. comprised in an intron, which is first processed by Drosha to form a pre-miRNA hairpin molecule. The pre-miRNA molecule is a shRNA-like molecule that can subsequently be processed by Dicer to result in an siRNA-like double stranded duplex. The miRNA, i.e. the guide strand that is part of the double stranded RNA duplex is subsequently incorporated in RISC. An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure, e.g. as predicted using e.g. m-fold software, the natural miRNA sequence as it is present in the RNA structure (i.e. duplex, pre-miRNA or pri-miRNA), and the sequence present in the structure that is complementary therewith are removed and replaced with a first RNA sequence, i.e. a polynucleotide in accordance with the invention, and a second RNA sequence according to the invention. The first RNA sequence and the second RNA sequence may be selected such that the RNA structures that are formed, i.e. pre-miRNA, pri-miRNA and/or miRNA duplex, resemble the corresponding predicted original sequences. Such pre-miRNA, pri-miRNA and miRNA duplexes (that consist of two separate RNA strands that are hybridized via complementary base pairing) as found in nature often are not fully base paired, i.e. not all nucleotides that correspond with the first and second strand as defined above are base paired, and the first and second strand are often not of the same length. How to use miRNA precursor molecules as scaffolds for any selected target sequence and substantially complementary first RNA sequence is described e.g. in Liu Y P Nucleic Acids Res. 2008 May; 36(9):281 1-24.

In any case, as is clear from the above, the double stranded RNA comprising the first and second RNA sequence, the first RNA sequence corresponding with the polynucleotide in accordance with the invention selected to sequence-specifically target misspliced transcripts, can comprise additional nucleotides and/or nucleotide sequences. The double stranded RNA may be comprised in a single RNA sequence or comprised in two separate RNA strands. Without being bound by theory, whatever design is used for the double stranded RNA, it is designed such that a sequence comprising the first RNA sequence, i.e. the polynucleotide of the invention, can be processed by the RNAi machinery such that it can be incorporated in the RISC complex to have its action. The said sequence comprising or consisting of the polynucleotide of the invention being capable of sequence-specifically targeting misspliced transcripts. Hence, as long as the double stranded RNA is capable of inducing RNAi, such a double stranded RNA is contemplated in the invention. Hence, in one embodiment, the double stranded RNA according to the invention is comprised in a pre-miRNA scaffold, a pri-miRNA scaffold, a shRNA, or an siRNA.

One endogenous miRNA, miR451 does not require Dicer for processing, but it is instead processed by the Argonaute 2 (Ago2) enzyme and subsequently trimmed by the Poly(A)-specific ribonuclease (PARN) to the mature 22/26-nt miR451 (Herrera-Carrillo and Berkhout, Nucleic Acids Res, 2017, 45(18):10369-10379). As shown in the examples, an artificial miRNA may be preferably incorporated in a pre-miRNA or a pri-miRNA scaffold derived from microRNA451a. The terms ‘microRNA451a’, ‘miR451’, ‘451 scaffold’ or simply ‘451’ are used interchangeably throughout this specification. This scaffold allows to induce RNA interference resulting in only guide strand induced RNA interference. The pri-miR451 scaffold does not result in a passenger strand because the processing is different from the canonical miRNA processing pathway (Cheloufi et al., Nature 2010 Jun. 3; 465(7298):584-9; Cifuentes et al, Science, 2010, 328 (5986), 1694-1698 and Yang et al., Proc Natl Acad Sci USA. 2010 Aug. 24; 107(34):15163-8). Hence, this miR451 scaffold represents a preferred embodiment of the invention, as unwanted potential off-targeting by passenger strands can be largely, if not completely, avoided.

As an alternative to the miR451 scaffold, similar Dicer independent structures may be preferably employed such as described herein and i.a. in Herrera-Carrillo and Berkhout, Nucleic Acids Res, 2017, Vol. 45 No.18 10369-79, which is incorporated herein by reference. As a passenger strand may result in off-targeting, e.g. targeting transcripts other than the desired target, using such a scaffold may allow one to avoid such unwanted targeting.

Whatever design is used for the miRNA scaffold, which is preferably based on miR451, it is designed such that therefrom an antisense RNA molecule comprising the first

RNA sequence, i.e. the sequence that replaced the original miRNA sequence and representing the polynucleotide in accordance with the invention that is to sequence-specifically target misspliced transcripts, in whole or a substantial part thereof, can be processed by the RNAi machinery such that it is incorporated in the RISC complex to have its action, i.e. to induce RNAi e.g. against the RNA target sequence comprised in an RNA encoded by a gene associated with a disease. The artificial miRNA that is produced from the miRNA scaffold is thus not necessarily identical in sequence length to the sequence that is used to replace the endogenous miRNA sequence. The artificial miRNA that is produced from the miRNA scaffold also not necessarily comprises the exact sequence that is used to replace the wild-type miRNA sequence. Thus, the miRNA sequence comprises or consists of the first RNA sequence, or the miRNA sequence comprises in whole or a substantial part of the first RNA sequence, said miRNA sequence being capable of sequence specifically targeting a gene, e.g. a gene transcript. Hence, as long as the miRNA produced from the miRNA scaffold is capable of inducing RNAi, such a scaffold is part of the invention. The artificial miRNA may thus preferably be comprised in a pre-miRNA scaffold or a pri-miRNA scaffold.

As shown in the examples, a polynucleotide in accordance with the invention (or first RNA sequence) of 22 nucleotides (e.g. for a miR451) in length may be selected and incorporated in a miRNA scaffold. Such a miRNA scaffold sequence is subsequently processed by the RNAi machinery as present in the cell. When reference is made to miRNA scaffold it is understood to comprise pri-miRNA structures or pre-miRNA structures.

miRNA scaffolds based on 451, when processed in a neuronal cell, can result in guide sequences, i.e. an artificial miRNA, comprising the polynucleotide in accordance with the invention (the (first RNA) sequence that replaced the endogenous 451 miRNA sequence) or a substantial part thereof, having a length which is in the range of 19-30 nucleotides as shown in the examples. Such guide strands are capable of reducing the target gene expression by targeting the selected target sequences. As is clear from the above, the polynucleotide sequence as it is encoded by the expression cassette of the invention, is comprised in part or in whole, in a guide strand when it has been processed by the RNAi machinery of the cell. Hence, the guide strand, i.e. artificial miRNA, that is to be generated from the RNA encoded by the expression cassette, comprising the first RNA sequence and the second RNA sequence is to comprise at least 18 nucleotides of the first RNA sequence. Preferably, such a guide strand comprises at least 19 nucleotides, 20 nucleotides, 21 nucleotides, or at least 22 nucleotides. A guide strand can comprise the polynucleotide sequence in accordance with the invention also as a whole. In selecting a miRNA scaffold e.g. from miRNA scaffolds as found in nature such as in humans, the polynucleotide sequence in accordance with the invention can be selected such that it is to replace the original guide strand. This does not necessarily mean that a guide strand produced from such an artificial scaffold are identical in length and sequence to the polynucleotide (or first RNA) sequence selected, nor may it necessarily be so that the polynucleotide sequence is in its entirety to be found in the guide strand that is produced.

A miRNA 451 scaffold, preferably comprises from 5′ to 3′, firstly 5′-CUUGGGAAUGGCAAGG-3′ (SEQ ID NO. 1), followed by a sequence of 22 nucleotides, comprising or consisting of the polynucleotide in accordance with the invention, followed by a sequence of 17 nucleotides, which is complementary over its entire length with nucleotides 2-18 of said sequence of 22 nucleotides, subsequently followed by sequence 5′-CUCUUGCUAUCCCAGA-3′ (SEQ ID NO. 2). Preferably the first 5′-C nucleotide of the latter sequence is not to base pair with the first nucleotide of the first RNA sequence. Such a scaffold may comprise further flanking sequences as found in the original pri-miR451 scaffold. Alternatively, the flanking sequences, may be replaced by flanking sequences of other pri-mRNA structures. It is understood that, as the miR451 scaffold can provide for guide strands only due to the length of the stem sequence, it is preferred that alternative flanking sequences do not extend the stem length of 17 consecutive base pairs. As is clear from the above, the sequence of the scaffold may differ not only with regard to the (putative) guide strand sequence, and sequence complementary thereto, as present in the wild-type scaffold, but may also comprise additional mutations in the 5′ sequence, loop sequence and 3′ sequence as well, as additional mutations may be required to provide for an RNA structure that is predicted to mimic the secondary structure of the wild-type scaffold and/or does not have a stem extending beyond 17 consecutive base pairs.

It is understood that the polynucleotide as comprised in a double stranded RNA that is to induce RNA interference in accordance with the invention can be administered to subjects suffering from a repeat expansion disease. The polynucleotide in accordance with the invention may also be expressed from an expression cassette. For example, the polynucleotide as comprised in a double stranded RNA that is to induce RNA interference in accordance with the invention can be expressed in a cell to thereby provide for durable reduction of misspliced transcripts. For example, a double stranded RNA can be expressed by convergent transcription, by expressing a shRNA sequence, or by expressing separate strands from separate expression cassettes. A double stranded RNA can also be comprised in a miRNA scaffold as described above, which may be part of a larger RNA transcript, e.g. a pol II expressed transcript, comprising e.g. a 5′ UTR and a 3′UTR and a poly A. Flanking structures may also be absent.

An expression cassette in accordance with the invention may thus express a shRNA-like structure having a sequence of 22 nucleotides, comprising or consisting of the polynucleotide in accordance with the invention, followed by a sequence of 17 nucleotides, which is complementary over its entire length with nucleotides 2-18 of said sequence of 22 nucleotides, and further comprising 1 or more additional nucleotides which is predicted not to form a base pair with the first RNA sequence. The latter shRNA-like structure being derived from the miR451 scaffold structure and it can be referred to as a pre-miRNA scaffold from miR451.

In a further embodiment, a polynucleotide for use in the treatment of an expanded repeat disorder in accordance with the invention is provided, wherein said misspliced transcript encodes a polyQ protein and wherein said polynucleotide induces a reduction of said polyQ protein encoded by said misspliced transcript. For example, CAG repeat expansions contained in frame within a misspliced transcript, e.g. within an exon sequence, when targeted, reduce the levels of said polyQ protein. Such a polyQ protein may also be a truncated polyQ protein, i.e. meaning that the amino acid sequence length is shorter as compared with a non-misspliced transcript. This is because, as said, missplicing can result in shorter transcripts that have polyadenylated from a cryptic polyadenylation site. Hence, in yet a further embodiment, the polynucleotide for use in the treatment of an expanded repeat disorder in accordance with the invention is provided, wherein said misspliced transcript encodes a truncated polyQ protein and wherein said polynucleotide induces a reduction of said truncated polyQ protein.

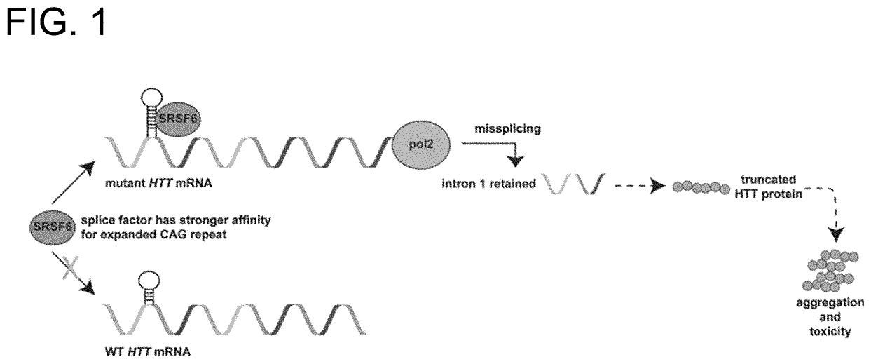

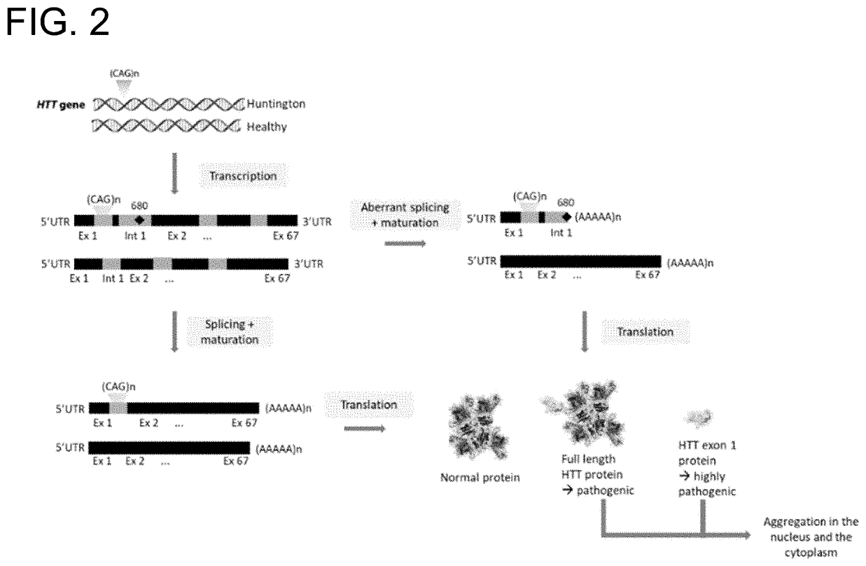

Examples of expanded repeat disorders that can produce a polyQ protein from a misspliced transcript include e.g. Huntington disease or Spinocerebellar Ataxia Type 3 (SCA3) (schematically depicted in FIGS. 1, 2 and FIG. 10). Genes having expanded repeats that cause disease may be referred to as mutant genes, producing mutant transcripts and mutant protein, e.g. in case of Huntington disease, one may refer to a mutant HTT gene, mutant HTT transcripts, and mutant HTT protein, likewise, in case of an expansion in ataxin-3 causing SCA3, one may refer to a mutant ataxin-3 gene, transcript or protein. Hence, reducing a transcript produced from a gene with a repeat expansion, i.e. causing a disease or disorder, may also be referred to as reducing a mutant transcript. In Huntington disease, the expanded repeat sequence allows for the utilization of a cryptic polyadenylation site within intron1, resulting in alternative transcripts which are misspliced, i.e. splicing is incomplete and intron 1 splicing does not occur. Truncated transcripts are formed which have terminated in a cryptic polyA signal within the intron sequence adjacent to the exon1 containing the expanded repeat. The truncated transcript is translated into a truncated poly Q protein, comprising the sequence of the exon with the expanded polyQ followed by the sequence encoded by the sequence of the adjacent intron (FIGS. 1 and 2).

In a further embodiment, the polynucleotide for use in the treatment of a repeat expansion disorder in accordance with the invention, includes the use in the treatment of Huntington Disease, said polynucleotide inducing a reduction of misspliced mutant HTT transcripts, wherein the exon with the CAG repeat expansion is exon 1 of mutant HTT and the intron sequence which is 3′ and adjacent therefrom is from intron 1 of mutant HTT. It is understood that said misspliced mutant HTT transcripts (or aberrant mutant HTT transcripts) have terminated in intron1. The transcript that is thus produced from a mutant HTT gene does not comprise exon 2 sequences or further HTT exon or intron sequences that are encoded by the HTT gene and are 3′ to the intron 1 encoding sequence. Such a misspliced mutant HTT transcript (or aberrant mutant HTT transcript) comprises an exon1 sequence with the expanded repeat and a part of Intron 1 until a cryptic polyA site. Examination of the genomic sequence for HTT intron 1 identified cryptic polyA sites at position 7327 bp (7.3 kb site) into HTT intron1 in the human genome. Cryptic polyA sites were also found located at position 680 bp and 1145 bp (1.2kb site) into HTT intron 1 in the mouse genome (Sathasivam 2013, Neueder 2018). Hence, such a misspliced transcript from a mutant Huntington gene does not comprise exon 2 or any downstream exons. Such a misspliced mutant HTT transcript is thus considerably shorter as compared to transcripts not misspliced. An exemplary DNA sequence encoding Exon1-Intron1 mRNA according to ensembl.org transcript HTT-201 (Human Transcript) ENST00000355072.10 is listed below. It comprises a human Exon 1 sequence containing 21 CAG repeats, Intron 1 sequence until cryptic polyA site located at 7327 bp (AATAAA, underlined) and further sequence until cleavage site and polyA tail. This sequence represents a sequence from an HTT gene that is not associated with disease, as it has 21 CAG repeats (in bold and underlined) (SEQ ID NO. 3).

GCTGCCGGGACGGGTCCAAGATGGACGGCCGCTCAGGTTCTGCTTTTACCTGCGG
CCCAGAGCCCCATTCATTGCCCCGGTGCTGAGCGGCGCCGCGAGTCGGCCCGAG
GCCTCCGGGGACTGCCGTGCCGGGCGGGAGACCGCCATGGCGACCCTGGAAAAG
CTGATGAAGGCCTTCGAGTCCCTCAAGTCCTTCCAGCAGCAGCAGCAGCAGCA
GCAGCAGCAGCAGCAGCAGCAGCAGCAGCAGCAGCAGCAACAGCCGCCACC
GCCGCCGCCGCCGCCGCCGCCTCCTCAGCTTCCTCAGCCGCCGCCGCAGGCACAG
CCGCTGCTGCCTCAGCCGCAGCCGCCCCCGCCGCCGCCCCCGCCGCCACCCGGCC
CGGCTGTGGCTGAGGAGCCGCTGCACCGACCGTGAGTTTGGGCCCGCTGCAGCT
CCCTGTCCCGGCGGGTCCCAGGCTACGGCGGGGATGGCGGTAACCCTGCAGCCT
GCGGGCCGGCGACACGAACCCCCGGCCCCGCAGAGACAGAGTGACCCAGCAAC
CCAGAGCCCATGAGGGACACCCGCCCCCTCCTGGGGCGAGGCCTTCCCCCACTTC
AGCCCCGCTCCCTCACTTGGGTCTTCCCTTGTCCTCTCGCGAGGGGAGGCAGAGC
CTTGTTGGGGCCTGTCCTGAATTCACCGAGGGGAGTCACGGCCTCAGCCCTCTCG
CCCTTCGCAGGATGCGAAGAGTTGGGGCGAGAACTTGTTTCTTTTTATTTGCGAG
AAACCAGGGCGGGGGTTCTTTTAACTGCGTTGTGAAGAGAACTTGGAGGAGCCG
AGATTTGCTCAGTGCCACTTCCCTCTTCTAGTCTGAGAGGGAAGAGGGCTGGGGG
CGCGGGACACTTCGAGAGGAGGCGGGGTTTGGAGCTGGAGAGATGTGGGGGCA
GTGGATGACATAATGCTTTTAGGACGCCTCGGCGGGAGTGGCGGGGCAGGGGGG
GGGCGGGGAGTGAGGGCGCGTCCAATGGGAGATTTCTTTTCCTAGTGGCACTTA
AAACAGCCTGAGATTTGAGGCTCTTCCTACATTGTCAGGACATTTCATTTAGTTC
ATGATCACGGTGGTAGTAACACGATTTTAAGCACCACCTAAGAGATCTGCTCATC
TAAGCCTAAGTTGGTCTGCAGGCGTTTGAATGAGTTGTGGTTGCCAAGTAAAGTG
GTGAACTTACGTGGTGATTAATGAAATTATCTTAAATATTAGGAAGAGTTGATTG
AAGTTTTTTGCCTATGTGTGTTGGGAATAAAACCAACACGTTGCTGATGGGGAGG
TTAATTGCCGAGGGATGAATGAGGTGTACATTTTACCAGTATTCCAGTCAGGCTT
GCCAGAATACGGGGGGTCCGCAGACTCCGTGGGCATCTCAGATGTGCCAGTGAA
AGGGTTTCTGTTTGCTTCATTGCTGACAGCTTGTTACTTTTTGGAAGCTAGGGGTT
TCTGTTGCTTGTTCTTGGGGAGAATTTTTGAAACAGGAAAAGAGAGACCATTAAA
ACATCTAGCGGAACCCCAGGACTTTCCCTGGAAGTCTGTGTGTCGAGTGTACAGT
AGGAGTTAGGAAGTACTCTGGTGCAGTTCAGGCCTTTCTCTTACCTCTCAGTATT
CTATTTCCGATCTGGATGTGTCCCAGATGGCATTTGGTAAGAATATCTCTGTTAA
GACTGATTAATTTTTAGTAATATTTCTTGTTCTTTGTTTCTGTTATGATCCTTGTCT
CGTCTTCAAAGTTTAATTAGAAAATGATTCGGAGAGCAGTGTTAGCTTATTTGTT
GGAATAAAATTTAGGAATAAATTATTCTAAAGGATGGAAAAACTTTTTGGATATT
TGGAGAAATTTTAAAACAATTTGGCTTATCTCTTCAGTAAGTAATTTCTCATCCAG
AAATTTACTGTAGTGCTTTTCTAGGAGGTAGGTGTCATAAAAGTTCACACATTGC
ATGTATCTTGTGTAAACACTAAACAGGGCTCCTGATGGGAAGGAAGACCTTTCTG
CTGGGCTGCTTCAGACACTTGATCATTCTAAAAATATGCCTTCTCTTTCTTATGCT
GATTTGACAGAACCTGCATTTGCTTATCTTCAAAATATGGGTATCAAGAAATTTC
CTTTGCTGCCTTGACAAAGGAGATAGATTTTGTTTCATTACTTTAAGGTAATATAT
GATTACCTTATTTAAAAAATTTAATCAGGACTGGCAAGGTGGCTTACACCTTTAA
TCCGAGCACTTTGGGAGGCCTAGGTGGACGAATCACCTGAGGTCAGGAGTTTGA
GACCAGCCTGGCTAACATGGTGAAACCCTGTCTCTACTAAAAATACAAAAATTA
GCTGGTCATGGTGGCACGTGCCTGTAATCCAAGCTACCTGGGAGGCTGAGGCAG
GAAAATCGCTTGAACCCGGGAGGCAGAGTCTGCAGTGAGTTGAGATCACGCCAC
TGCACTCCAGCCTGGGTGACAGAGCGAGACTCTATCTCAAAAAAAATTTTTTTTA
ATGTATTATTTTTGCATAAGTAATACATTGACATGATACAAATTCTGTAATTACA
AAAGGGCAATAATTAAAATATCTTCCTTCCACCCCTTTCCTCTGAGTACCTAACTT
TGTCCCCAAGAACAAGCACTATTTCAGTTCCTCATGTATCCTGCCAGATATAACC
TGTTCATATTGTAAGATAGATTTAAAATGCTCTAAAAACAAAAGTAGTTTAGAAT
AATATATATCTATATATTTTTTGAGATGTAGTCTCACATTGTCACCCAGGCTGGAG
TGCAGTGATACAATCTCGGCTCACTGCAGTCTCTGCCTCCCAGGTTCAAATGCTT
CTCCTGCCTCAGCCTTCTGAGTAGCTGGGATTACAGGCGCCCACCACCATGTCCA
GCTAATTTTTGTATTTTTAGTAGAGATGGGGTTTCACCATGTTGGCCAGGCTGGTC
TTGAACTCCTGACCTTGTGATCTGTCCACCTCGGCCTCCCAAAGTGCTGGGATTA
CAGGTGTGAGCCACCATGCCTGGCTAGAATAATAACTTTTAAAGGTTCTTAGCAT
GCTCTGAAATCAACTGCATTAGGTTTATTTATAGTTTTATAGTTATTTTAAATAAA
ATGCATATTTGTCATATTTCTCTGTATTTTGCTGTTGAGAAAGGAGGTATTCACTA
ATTTTGAGTAACAAACACTGCTCACAAAGTTTGGATTTTGGCAGTTCTGTTCACG
TGCTTCAGCCAAAAAATCCTCTTCTCAAAGTAAGATTGATGAAAGCAATTTAGAA
AGTATCTGTTCTGTTTTTATGGCTCTTGCTCTTTGGTGTGGAACTGTGGTGTCACG
CCATGCATGGGCCTCAGTTTATGAGTGTTTGTGCTCTGCTCAGCATACAGGATGC
AGGAGTTCCTTATGGGGCTGGCTGCAGGCTCAGCAAATCTAGCATGCTTGGGAG
GGTCCTCACAGTAATTAGGAGGCAATTAATACTTGCTTCTGGCAGTTTCTTATTCT
CCTTCAGATTCCTATCTGGTGTTTCCCTGACTTTATTCATTCATCAGTAAATATTT
ACTAAACATGTACTATGTGCCTGGCACTGTTATAGGTGCAGGGCTCAGCAGTGAG
CAGACAAAGCTCTGCCCTCGTGAAGCTTTCATTCTAATGAAGGACATAGACAGTA
AGCAAGATAGATAAGTAAAATATACAGTACGTTAATACGTGGAGGAACTTCAAA
GCAGGGAAGGGGATAGGGAAATGTCAGGGTTAATCGAGTGTTAACTTATTTTTAT
TTTTAAAAAAATTGTTAAGGGCTTTCCAGCAAAACCCAGAAAGCCTGCTAGACA
AATTCCAAAAGAGCTGTAGCACTAAGTGTTGACATTTTTATTTTATTTTGTTTTGT
TTTGTTTTTTTTGAGACAGTTCTTGCTCTATCAGCCAGGCTGGAGTGCACTAGTGT
GATCTTGGCTCACTGCAACCTCTGCCTCTTGGGTTCAAGTGATTCTCATGCCTCAG
CCTCCTGTTTAGCTGGGATTATAGACATGCACTGCCATGCCTGGGTAATTTTTTTT
TTTTCCCCCGAGACGGAGTCTTGCTCTGTCGCCCAGGCTGGAGTGCAGTGGCGCG
ATCTCAGCTCACTGCAAGCTCCGCTTCCCGAGTTCACGCCATTCTCCTGCCTCAGT
CTCCCAAGTAGCTGGGACTACAGGCGCCTGCCACCACGTCCAGCTAATTTTTTTG
TATTTTTAATAGAGACGGGGTTTCACCGTGTTAGCCAGGATGATCTTGATCTCCT
GACCTCGTCATCCGCCGACCTTGTGATCCGCCCACCTCGGCCTCCCAAAGTGCTG
GGATTACAGGCATGAGCCACTGTGCCCGGCCACGCCTGGGTAATTTTTGTATTTT
TAGTAGAGATGGGGTTTTGCCATGATGAGCAGGCTGGTCTCGAACTCCCGGCCTC
ATGTGATCTGCCTGCCTTGGCCTCCCAAAGTGCTAGGATTACAGGCATGAGCCAC
CATACCTGGCCAGTGTTGATATTTTAAATACGGTGTTCAGGGAAGGTCCACTGAG
AAGACAGCTTTTTTTTTTTTTTTTTTTGGGGTTGGGGGGCAAGGTCTTGCTCTTTA
ACCCAGGCTGGAATGCAGTATCACTATCGTAGCTCACTTCAGCCTTGAACTCCTG
GGCTCAAGTGATCCTCCCACCTCAACCTCACAATGTGTTGGGACTATAGGTGTGA
GCCATCACACCTGGCCAGATGATGGCTTTTGAGTAAAGACCTCAAGCGAGTTAA
GAGTCTAGTGTAAGGGTGTATGAAGTAGTGGTATTCCAGATGGGGGGAACAGGT
CCAAAATCTTCCTGTTTCAGGAATAGCAAGGATGTCATTTTAGTTGGGTGAATTG
AGTGAGGGGGACATTTGTAGTAAGAAGTAAGGTCCAAGAGGTCAAGGGAGTGCC
ATATCAGACCAATACTACTTGCCTTGTAGATGGAATAAAGATATTGGCATTTATG
TGAGTGAGATGGGATGTCACTGGAGGATTAGAGCAGAGGAGTAGCATGATCTGA
ATTTCAATCTTAAGTGAACTCTGGCTGACAACAGAGTGAAGGGGAACACCGGCA
AAAGCAGAAACCAGTTAGGAAGCCACTGCAGTGCTCAGATAAGCATGGTGGGTT
CTGTCAGGGTACCGGCTGTCGGCTGTGGGCAGTGTGAGGAATGACTGACTGGAT
TTTGAATGCGGAACCAACTGCACTTGTTGAACTCTGCTAAGTATAACAATTTAGC
AGTAGCTTGCGTTATCAGGTTTGTATTCAGCTGCAAGTAACAGAAAATCCTGCTG
CAATAGCTTAAACTGGTAACAAGCAAGAGCTTATCAGAAGACAAAAATAAGTCT
GGGGAAATTCAACAATAAGTTAAGGAACCCAGGCTCTTTCTTTTTTTTTTTTTTGA
AACGGAGTTTCGCTCTTGTCACCCGGGCTGGAGTGCAATGATGTGATCTCAGCTC
ACTAAAACCTCTACCTCCTGGGTTCAAGTGATTCTTCTGCCTCAGCCTCCCAAGT
AACTGGGATTACAGGCGTATACCACCATGCCCAGCTAATTTTTGTGTTTTTAGTA
GAGATGGGGTTTCACCATGTTGGCCAGGCTGGTCTCGAACTTCTGACCTCAGGTG
ATCCACTCGCCTCAGCCTGCCAAAGTGCTGGGATTACAGGTTTGGGCCACTGCAC
CCGGTCAGAACCCAGGCTCTTTCTTATACTTACCTTGCAAACCCTTGTTCTCATTT
TTTCCCTTTGTATTTTTATTGTTGAATTGTAATAGTTCTTTATATATTCTGGATACT
GGATTCTTATCAGATAGATGATTTGTAAAAACTCTCCCTTCCTTTGGATTGTCTTT
TTACTTTCTTGATAGTGTCTTTTGAAGTGTAAAAGTTTTTAATTTTGATGAAGTCG
AGTTTATCTATTTTGTCTTTGGTTGCTGTGCTTCAAGTGTCATATCTAAGAAATCA
TTGTCTAATCCAAAGTCAAAAAGGTTTACTCCTATGTTTTCTTCTAAGAATTTTAG
AGTTTTACATTTAAGTCTGATCCATTTTGAGTTAATTTTTATATATGGTTCAGGTA
GAAGTCCAACTTTATTCTTTTCCATGTGGTTATTCAGTTGTCCCAGCACTGTTTGT
TGAAGAGACTATTCTTTCCCCATGGAATTATCTTAGTACCCTTGTTGAAAATTAAT
CGTCCTTAATTGTATAAATTTATTTCTAGACTGTCAGTTCTACCTGTTGGTCTTTAT
GTCGATCCTGTGCCAGTACCATACAGTCTTGATTACTGAAGTTTGTGTCACAGTTT
AAATTCATGAAATGTGAGTTCTCCAACTTTGTTCCTTTTCAAGATTGATTTGGCCA
TGCTGGGTCCCTTGCATTTCCGTACGAATTGTAGGATCAGCTTGTCAGTTTCAAC
AAAGAAGCCAAGTAGGATTCTGAGAGGGATTGTGTTGAATCTGTAGATCAACTT
GGGGAGTATTCGCATCTTAACAATATTGTCTTCCACCTATGAACATGGGCAAACT
TTGTGTAAATGGTCAGATTGTAAGTATTTCGGGCTGTGTGGGCACAGTGTCTCTG
TCACAGCTACGCGGCTCTGCCATTGTAGCATGAAAGTAGCCATAAGCAATATGTA
TGAGTGTCTGTGTTCCAATAGAATTTTATTAATGACAAGGAAGTTTGAATTTCAT
ATAATTTTCACCTGTCATGAGATAGTATTTGATTATTTTGGTCAACCATTTAAAAA
TGTAAAAACATTTCTTAGCTTGTGAACTAGCCAAAAATATGCAGGTTATAGTTTT
CCCACTCCTAGGTTAAAATATGATAGGACCACATTTGGAAAGCATTTCTTTTTTTT
TTTTTTTTTTTTTTTTTGAGACGGAGTTTCACTCTTGTTGCCCAGGCTGGAGTGCA
GTGGCGCGATCTCGGCTCACTGCAACCTCTGCCTCCCAGGTTCAAGACATTCTCC
TGCACGGCCTCCCTAGTAGCTGGGATTACAGGCATGCGCCACCACACCCAGCTA
ATTTTGTATTTTTAGTAGAGACGGGGTTTCTCCATGTTGGTCAGGCTGGTCTTGAA
CTCCTGACCTCAGGTGATCCACCCGCCTCAGCCTCCCAAAGTGCTGGGATTACAG
GGTGTGAGCCACCACACCCTGCTGGAAAGCATTTCTTTTTTGGCTGTTTTTGTTTT
TTTTTTAAACTAGTTTTGAAAATTATAAAAGTTACACATATACATTATAAAAATA
TCTTCAAGCAGCACAGATGAAAAACAAAGCCCTTCTTGCAAGTCTGTCATCTTTG
TCTAACTTCCTAAGAACAAAAGTGTTTCTTGTGTCTTCTTCCCAGATTTTAATATG
CATATACAAGCATTTAAATGTGTCATTTTTTGTTTGCTTGACTGAGATCACATTAC
ATATGTATTTTTTTACTTAACAATGTGTCATAGATATTGTTCCATAGCAGTACCTG
TAATTCTTATTAATTGCTATGTAATATTTTAGAATTTCTTTTTAAAAGAGGACTTT
TGGAGATGTAAAGGCAAAGGTCTCACATTTTTGTGGCTGTAGAATGTGCTGGTGA
CATATTCTCTCTACCTTGAGAAGTCCCCATCCCCATCACCTCCATTTCCTGTAAAT
AAGTCAACCACTTGATAAACTACCTTTGAATGGATCCACACTCAAAACATTTAGT
CTTATTCAGACAACAAGGAGGAAAAATAAAATACCTTATAAAGCAAAAAAAA...

Preferably said polynucleotide in accordance with the invention targets a sequence within a misspliced transcript of mutant HTT. Such a misspliced mutant HTT transcript can be a sequence comprising the encoded exemplary nucleotide sequence above, having instead of the 21 CAG repeats a disease-causing repeat expansion, i.e. more than 35 CAG repeats, or a corresponding sequence thereof. It is understood that a sequence corresponding therewith includes natural polymorphisms within said mutant HTT sequence, and includes sequences having a different CAG repeat sequences (corresponding to bold and underlined nucleotides of the listed sequence above), as the CAG repeat regions vary between Huntington patients (a CAG repeat region of more than 35 codons). It is understood that said exemplary sequence above represents DNA. The encoded mRNA is represented by the same sequence but lists instead of a U (uracil) a T (thymine). Preferably, said target sequence is either 3′ or 5′ from said expanded repeat sequence within the misspliced mutant HTT transcript. More preferably, said target sequence is selected to be 5′ from said expanded repeat sequence. This may be preferred because advantageously selecting a sequence 5′ from said expanded repeat sequence allows one to target both misspliced transcripts and canonical transcripts that might contribute to disease pathology. Said target sequence preferably being selected from a sequence of an exon, e.g. selected from

(SEQ ID NO. 4)
5′-GCUGCCGGGACGGGUCCAAGAUGGACGGCCGCUCAGGUUCUGCUUU
UACCUGCGGCCCAGAGCCCCAUUCAUUGCCCCGGUGCUGAGCGGCGCCG
CGAGUCGGCCCGAGGCCUCCGGGGACUGCCGUGCCGGGCGGGAGACCGC
CAUGGCGACCCUGGAAAAGCUGAUGAAGGCCUUCGAGUCCCUCAAGUCC
UUC-3′

(Sequence 5′ from expanded CAG repeat in Exon1 of HTT, position 1-196). Preferably said polynucleotide that targets said sequence targets a sequence corresponding or overlapping with 5′-GAGACCGCCAUGGCGACCCUGGA-3′ (SEQ ID NO. 5), 5′-AGACCGCCAUGGCGACCCUGGAA-3′ (SEQ ID NO. 6) or 5′-GAUGAAGGCCUUCGAGUCCCUCAA-3′ (SEQ ID NO. 7). More preferably said polynucleotide that targets said sequence targets a sequence corresponding with 5′-GGCCUUCGAGUCCCUCAAGUCCUU-3′ (SEQ ID NO. 8) (ensembl.org transcript HTT-201 (Human Transcript) ENST00000355072.10 Exon1 mRNA position 172-195). Most preferably said polynucleotide targeting said target sequence 5′-GGCCUUCGAGUCCCUCAAGUCCUU-3′ (SEQ ID NO. 8) (ensembl.org transcript HTT-201 (Human Transcript) ENST00000355072.10 Exon1 mRNA position 172-195) comprises or consists of 5′-AAGGACUUGAGGGACUCGAAGA-3′ (SEQ ID NO. 9) or 5′-AAGGACUUGAGGGACUCGAAG-3′ (SEQ ID NO. 10). It is understood that said sequences represent RNA. The corresponding DNA comprises the same sequence but instead of a U (uracil) list a T (thymine). Said polynucleotide preferably being comprised in a miRNA scaffold, such as a miRNA scaffold derived from miR451, and as described in WO2016102664 (incorporated herein by reference) and as described in the examples. Preferably said miRNA scaffold comprises 5′-AAGGACUUGAGGGACUCGAAGA-3′ (SEQ ID NO. 9).

In another or further embodiment, the invention provides for a polynucleotide for use in the treatment of a CAG repeat disorder in accordance with the invention, wherein said misspliced HTT transcripts that are reduced encode a highly pathogenic truncated polyQ HTT protein and wherein said polynucleotide induces in a reduction of said truncated polyQ HTT protein. Said misspliced HTT transcripts are translated in a truncated polyQ HTT protein, said translation terminating being in the intron 1 sequence and the truncated polyQ HTT protein may also be referred to as pathogenic N-terminal HTT protein. Said truncated polyQ HTT protein may have an amino acid sequence such as listed below MATLEKLMKAFESLKSFQQQQQQQQQQQQQQQQQQQQQPPPPPPPPPPPQLPQPPPQ AQPLLPQPQPPPPPPPPPPGPAVAEEPLHRP (SEQ ID NO. 11), having different lengths of the polyQ repeat (corresponding to the underlined Q stretch of said listed amino acid sequence), as the lengths of polyQ repeats vary between Huntington patients, or an amino acid sequence corresponding therewith. Said truncated polyQ HTT protein corresponding to the sequence encoded by exon1 (due to the presence of an in-frame stop codon after the first nucleotide of intron1). Therefore, said truncated polyQ HTT protein does not contain any amino acid sequence derived from Intron1 nucleotide sequence. It is understood that corresponding amino acid sequences include natural polymorphisms of such truncated HTT proteins.

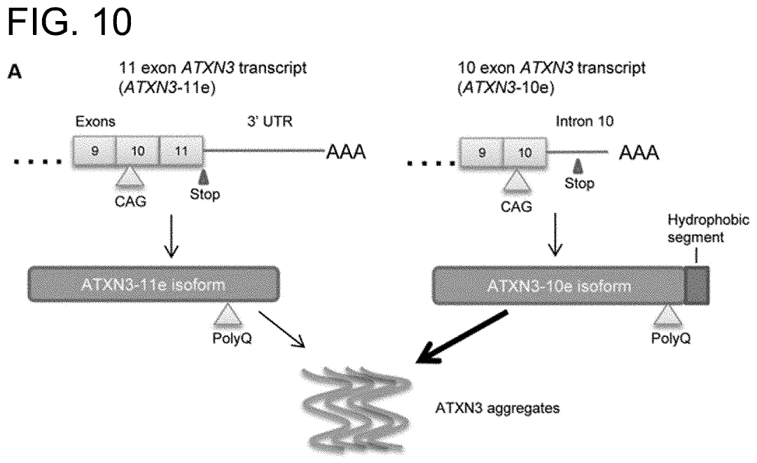

In another further embodiment, the polynucleotide for use in the treatment of a repeat expansion disorder in accordance with the invention is for use in the treatment of SCA3, said polynucleotide inducing a reduction of misspliced ataxin-3 transcripts (see FIG. 10). Said ataxin-3 misspliced transcripts comprise at its 3′ end exon 10, which comprises the disease-causing expanded CAG repeat and an intron 10 sequence which is 3′ and adjacent therefrom. Ataxin-3 transcripts that are expressed from the ataxin-3 gene comprise many splice variants, having different exon compositions. Missplicing or aberrant splicing from expressed transcripts from the ataxin-3 gene provide for further transcripts that can be targeted in accordance with the invention. Such aberrant transcripts may comprise the same exon composition variation as observed upstream from exon 10 as observed for transcripts that are not misspliced. Aberrant ataxin-3 transcripts have a different sequence 3′ from exon 10, i.e. instead of having exon 11 and a subsequent 3′ UTR sequence, such aberrant ataxin-3 transcripts comprise a sequence derived from intron 10, encoded by the intron 10 sequence, which, like the HTT gene, also comprises a cryptic poly A signal. Such transcripts, when translated into protein, may not encode an amino acid C-terminus sequence that corresponds with the sequence encoded by exon 11, but encode a hydrophobic segment encoded by intron 10 instead, which may accelerate mutant ATXN3 aggregation (FIG. 10).

The DNA sequence encoding exon 10 is

(SEQ ID NO. 12)
ACAGCAGCAAAAG [symbol] [symbol] GGGGACCT
ATCAGGACAGAGTTCACATCCATGTGAAAGGCCAGCCACCAGTTCAGGAGC
ACTTGGGAGTGATCTAG.

The sequence encoding exon 11 is: GTGATGCTATGAGTGAAGAAGACATGCTTCAGGCAGCTGTGACCATGTCTTTAGA AACTGTCAGAAATGATTTGAAAACAGAAGGAAAAAAATAA (SEQ ID NO. 13). In this example, the sequence of exon 10 does not contain an expanded repeat sequence that causes disease. It is understood that mutant ataxin-3 genes that can cause disease all have an expanded repeat sequence that is at least 45 CAG repeats in length.

When spliced in non-disease genes, this results in a canonical protein sequence from the expanded repeat sequence onwards of (Q)n

(SEQ ID NO. 14)
GDLSGQSSHPCERPATSSGALGSDLGDAMSEEDMLQAAVTMSLETVRNDLK
TEGKK.

The underlined sequence encoded by exon 11. When missplicing occurs in diseased genes, in mutant ataxin-3 genes having expanded repeats encoding at least 45 glutamines, the sequence encoding exon 11 is not spliced to exon 10, and instead a sequence derived from intron 10 remains and transcription terminates at a cryptic poly A signal within intron 10. Said intron 10 derived sequence corresponding with GTAAGGCCTGCTCACCATTCATCATGTTCGCTACCTTCACACTTTATCTGACATAC GAGCTCCATGTGATTTTTGCTTTACATTATTCTTCATTCCCTCTTTAATCATATTAA GAATCTTAAGTAAATTTGTAATCTACTAAATTTCCCTGGATTAAGGAGCAGTTAC CAAAAGAAAAAAAAAAAAAAAA (SEQ ID NO. 15). When protein is produced from said exon 10 sequence and subsequent intron 10 sequence, a mutant ataxin-3 protein is produced having the following amino acid sequence at its C-terminus: ((Q)n representing the polyglutamine repeat):

(SEQ ID NO. 16)
(Q)n GDLSGQSSHPCERPATSSGALGSDLGKACSPFEWATFTLYLTYELH
VIFALHYSSFPL.

The underlined sequence encoded by the intron 10 derived sequence. This C-terminus protein sequence encoded by intron 10 is a hydrophobic segment that may accelerate mutant ATXN3 aggregation and contribute to pathology.

Hence, it is understood that said misspliced mutant ataxin-3 transcripts (or aberrant mutant ataxin-3 transcripts) have terminated in intron10. The transcript that is thus produced from mutant ataxin-3 does not comprise an exon 11 sequence that is encoded by the ataxin-3 gene. Such a misspliced mutant ataxin-3 transcript comprises an exon10 sequence with the expanded repeat and a part of intron 10 until cryptic polyA site.

Preferably said polynucleotide in accordance with the invention targets a sequence within a misspliced transcript of mutant ataxin-3. Such a misspliced mutant ataxin-3 transcript is a sequence encoded by the exemplary nucleotide sequence above, having instead of the 10 CAG repeats a disease-causing repeat expansion, i.e. more than 45 CAG repeats, or a corresponding sequence thereof. It is understood that a sequence corresponding therewith includes natural polymorphisms within said ataxin-3 sequence, and includes sequences having different CAG repeat sequences (corresponding to bold and underlined nucleotides of the listed sequence above), as the CAG repeat regions vary between SCA3 patients. It is understood that said exemplary sequence above represents DNA. The encoded mRNA is represented by the same sequence but lists instead of a U (uracil) a T (thymine). Preferably, said target sequence is either 3′ or 5′ from said expanded repeat sequence within the misspliced ataxin-3 transcript. More preferably, said target sequence is selected to be 5′ from said expanded repeat sequence. This may be preferred because advantageously selecting a sequence 5′ from said expanded repeat sequence allows one to target both misspliced transcripts and transcripts that have not misspliced. Said target sequence preferably being selected from a sequence of an exon. Preferably, said target sequences comprising a sequence selected from 5′-AACACUGGUUUACAGUUAGAAA-3′ (SEQ ID NO. 17), 5′-AAUUAGGAAAACAGUGGUUUAA-3′ (SEQ ID NO. 18), 5′-AAGUAUGCAAGGUAGUUCCAGA-3′ (SEQ ID NO. 19), 5′-UACUUCAGAAGAGCUUCGGAAG-3′ (SEQ ID NO. 20), or 5′-GAGACGAGAAGCCUACUUUGAA-3′ (SEQ ID NO. 21). Preferably said polynucleotide sequence targeting mutant misspliced ataxin-3 transcripts sequence specifically targets said misspliced transcripts via RNA interference. Preferably, said polynucleotide sequence comprises a sequence selected from 5′- UUUCUAACUGUAAACCAGUGUU-3′ (SEQ ID NO. 22), 5′- UUAAACCACUGUUUUCCUAAUU-3′ (SEQ ID NO. 23), 5′-UCUGGAACUACCUUGCAUACUU-3′ (SEQ ID NO. 24), 5′-CUUCCGAAGCUCUUCUGAAGUA-3′ (SEQ ID NO. 25) or 5′-UUCAAAGUAGGCUUCUCGUCUC-3′ (SEQ ID NO. 26). Said polynucleotide preferably being comprised in a miRNA scaffold, such as a miRNA scaffold derived from miR451, such as described in WO2016102664 and such as described in the examples.

In another or further embodiment, the invention provides for a polynucleotide for use in the treatment of a CAG repeat disorder in accordance with the invention, wherein said misspliced mutant ataxin-3 transcripts that are reduced encode a mutant ataxin-3 protein and wherein said polynucleotide induces in a reduction of said mutant ataxin-3 protein. Such protein comprising at its C-terminus not an amino acid sequence encoded by exon 11. Such a mutant ataxin-3 protein comprising at its C-terminus an amino acid sequence encoded by intron 10. Such a C-terminus of a mutant ataxin-3 protein may have the following amino acid sequence, (Q)n

(SEQ ID NO. 16)
GDLSGQSSHPCERPATSSGALGSDLGKACSPFIMFATFTLYLTYELHVIFA
LHYSSFPL,

having different lengths of the polyQ repeat (corresponding to the underlined (Q)n stretch of said listed amino acid sequence), as the lengths of polyQ repeats vary between SCA3 patients, or an amino acid sequence corresponding therewith (such as natural polymorphism).

As said, in accordance with the invention, and without being bound by theory, polynucleotides in accordance with the invention may be provided for expansion repeat disorders wherein said repeat expansion disorder results in missplicing 3′ from said repeat expansion, and wherein said polynucleotide is capable of inducing a reduction of said misspliced transcripts, e.g. via sequence specific inhibition, such as RNA interference. Preferably, such diseases with repeat expansion disorders are repeat expansion disorders that occur in exon sequences (as shown i.a. in the examples). Expansion repeat disorders that occur within exon sequences can be polyglutamine, polyalanine, or polyaspartic acid repeat disorders. Polyglutamine expansion disorders for which polynucleotides in accordance with the invention may be provided are Dentatorubral-Pallidoluysian Atrophy (DRPLA) (expansion within the ATN1 gene), Huntington Disease (HD) (expansion within the HTT gene), Spinal and Bulbar Muscular Atrophy (SBMA) (expansion within the Androgen Receptor gene), SCA type 1 (SCA1) (expansion within the ATXN1 gene), SCA type 2 (SCA2)—(expansion within the ATXN2 gene), SCA type 3 (SCA3)—(expansion within the ATXN3 gene), SCA type 6 (SCA6)—(expansion within the CACNA1A gene), SCA type 7 (SCA7) (expansion within the ATXN7 gene), SCA type 8 (SCA8) (expansion within the ATXN8 gene), SCA type 17 (SCA17) (expansion within the BP gene). Polyalanine disorders for which polynucleotides in accordance with the invention may be provided are Blepharophimosis Syndrome (BPES) (expansion within the FOXL2 gene), Cleidocranial Dysplasia (CCD) (expansion within the RUNX2 gene), Congenital Central Hypoventilation

Syndrome (CCHS) (expansion within the PHOX2B gene), Hand-Foot-Genital Syndrome (HFGS) (expansion within the HOXA13 gene), Holoprosencephaly (HPE) (expansion within the ZIC2 gene), Oculopharyngeal Muscular Dystrophy (OPMD)—(expansion within the PABPN1 gene), Synpolydactyly Syndrome (SPD) (expansion within the HOXD3 gene), X-linked Mental Retardation and Abnormal Genitalia (XLAG) and X-linked Mental Retardation (XLMR) (expansion within the ARX gene), XLMR and Growth Hormone Deficit (XLMRGHD) (expansion within the SOX3 gene). A polyspartic acid expansion disorders for which which polynucleotides in accordance with the invention may be provided are Pseudoachondroplasia and Multiple Epiphyseal Dysplasia (PSACH/MED)—(expansion within the COMP gene).

Preferably, the polynucleotide for use in accordance with the invention is encoded by a gene delivery vector for use in providing the polynucleotide. Such a gene delivery vector comprising a nucleotide sequence with an expression cassette encoding the polynucleotide in accordance with the invention. Hence, in another embodiment, a gene delivery vector is provided encoding a polynucleotide in accordance with the invention for use in the treatment of a repeat expansion disorder. Preferably, said gene delivery vector is for use in the treatment of Huntington's disease, as exemplified e.g. in the example section.

There are many known gene delivery vectors, both viral and non-viral. Said gene delivery vector is to comprise an expression cassette comprising the nucleic acid encoding the polynucleotide in accordance with the invention. Preferably, gene delivery vectors are used that can stably transfer the nucleic acid and/or expression cassette to cells in a human patient such that expression of the polynucleotide can be achieved. Suitable vectors may be lentiviral vectors, retrotransposon-based vector systems, or adeno-associated viral (AAV) vectors. It is understood that as e.g. lentiviral vectors carry an RNA genome, the RNA genome (a nucleic acid) will encode for the said expression cassette such that after transduction of a cell and reverse transcription a double stranded DNA sequence is formed comprising the nucleic acid sequence and/or said expression cassette in accordance with the invention.

All such vectors are within the scope of the present invention, but the preferred vector is based on an AAV vector. AAV sequences that may be used in the present invention for the production of AAV vectors, e.g. as produced in insect or mammalian cell lines, can be derived from the genome of any AAV serotype. The production of AAV vectors comprising an expression cassette of interest is described i.a. in; WO2007/046703, WO2007/148971, WO2009/014445, WO2009/104964, WO2011/122950, WO2013/036118, which are incorporated herein in its entirety. Generally, the AAV serotypes have genomic sequences of significant homology at the amino acid and the nucleic acid levels, provide an identical set of genetic functions, produce virions, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 may be a preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and/or VP3 capsid proteins for use in the context of the present invention may preferably be taken from AAV1, AAV2, AAV3, AAV4, AAV5, AAV7, AAV8, AAV9, AAVrh10 and AAV10 as these are serotypes that are suitable for use in gene therapy, such as for the treatment of the CNS. Also, newly developed AAV-like particles obtained by e.g. capsid shuffling techniques and AAV capsid libraries comprising mutations (insertions, deletions, substitutions), derived from AAV capsid sequences, and selected from such libraries as being suitable for specific target tissue transduction may be contemplated. AAV capsids may consist of VP1, VP2 and VP3 capsid proteins, but may also consist of VP1 and VP3 capsid proteins. AAV capsids may not contain any substantial amount of VP2 capsid protein. This is because the VP2 capsid protein may not be essential for efficient transduction.

A preferred AAV vector that may be used in accordance with the invention is an AAV vector of serotype 5. AAV of serotype 5 (also referred to as AAV5) has been shown useful for many tissue types and has been shown to be particularly useful for transducing human neuronal cells. AAV vectors comprising AAV5 capsids can comprise AAV5 VP1, VP2 and VP3 capsid proteins. AAV vectors comprising AAV5 capsids can also comprise AAV5 VP1 and VP3 capsid proteins, while not comprising AAV5 VP2 capsid proteins or at least not comprising any substantial amount of VP2 capsid proteins. In a wild-type derived AAV5 capsid protein, the VP1, VP2 and VP3 capsid proteins comprise identical amino acid sequences at their C-termini. The VP3 sequence is comprised in the VP2 sequence, and the VP2 sequence is comprised in the VP1 sequence. The N-terminal part of the VP1 amino acid sequence that is not contained in the VP2 and VP3 capsid proteins is positioned at the interior of the virion. This N-terminal VP1 sequence may e.g. be exchanged with an N-terminal sequence of another serotype, e.g. from serotype 2, whereas the VP2 and VP3 amino acid sequences may be entirely based on the AAV5 serotype. Such non-natural capsids comprising hybrid VP1 sequences, and such hybrid vectors are also understood to be AAV5 viral vectors in accordance with the invention. Such a hybrid vector of the AAV5 serotype is i.a. described by Urabe et al., J Virol. 2006. Furthermore, AAV5 capsid sequences may also have one or more amino acids inserted or replaced to enhance manufacturing and/or potency of a vector, such as i.a. described in WO2015137802. Such modified AAV5 capsids are also understood to be also of the AAV5 serotype.

AAV (also referred to as AAV vector) is preferred because it may remain episomal for a long time, thus giving prolonged expression, but having a very low integration frequency into the host genome, with a very low risk of undesired integration at undesired sites. As the preferred gene delivery vehicles are intended to treat diseases of the brain, the invention has as a preferred embodiment a method wherein said miRNA expressed in the brain is expressed through the introduction of a gene delivery vehicle in the brain. A preferred route of administration of AAV may be to the cerebrospinal fluid (CSF), i.e. intrathecally, such as described e.g. in WO2015060722 or Watson, et al., Gene Therapy, 2006. Another preferred route of administration of AAV may be via intra-striatal administration. Intrastriatal administration can be done by convection enhanced delivery using micro step-cannulae and real time MRI guidance. Intrathecal and intrastriatal administration may also be combined. An alternative route of administration may be intraparenchymal or subpial administration.

The polynucleotide to be delivered according to the invention is preferably comprised in a 451 scaffold. The miRNA451 scaffold has been disclosed in WO2011133889 and WO2016102664. It has as one of its advantages that is does not generate passenger strand, but more importantly, the present inventors have shown that it can be used as a scaffold to generate artificial miRNAs that can efficiently reduce misspliced transcripts, thereby making its use in the present invention preferred.

Therefore, the invention provides a gene delivery vector for use in accordance to the invention, wherein said gene delivery vector is a virus derived particle, most preferably wherein said gene delivery vehicle is an AAV based particle. AAV-based gene delivery of polynucleotides of the invention comprised in the miR451 scaffold are denoted as AAV-miQURE. For example, AAV-based gene delivery of a polynucleotide targeting the huntingtin gene that is associated with Huntington Disease and comprised in the miR451 scaffold is denoted as AAV-miHTT. Similarly, AAV-based gene delivery of a polynucleotide targeting the ataxin-3 gene that is associated with Spinocerebellar Ataxia Type 3 and comprised in the miR451 scaffold is denoted as AAV-miATXN. AAV has a set of features that makes it particularly suitable for gene therapy (see Naso et al), including the long-time maintenance of expression in target cells without viral material integrating in the host cell genome (possibly in harmful places).

As used in the description of the invention, clauses and clauses appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

### EMBODIMENTS

1. A polynucleotide for use in the treatment of a repeat expansion disorder, wherein said repeat expansion disorder results in missplicing 3′ from said repeat expansion, producing a misspliced transcript, and wherein said polynucleotide is capable of inducing a reduction of said misspliced transcript.
2. A polynucleotide for use in the treatment of a repeat expansion disorder, wherein said said repeat expansion is a CAG repeat, wherein said repeat expansion disorder results in missplicing 3′ from said repeat expansion, producing a misspliced transcript, and wherein said polynucleotide is capable of inducing a reduction of said misspliced transcript.
3. A polynucleotide for use in the treatment of a repeat expansion disorder according to embodiment 2, said misspliced transcripts containing an exon comprising the CAG repeat and containing an intron sequence which is 3′ and adjacent from said exon with the CAG repeat.
4. A polynucleotide for use in the treatment of a repeat expansion disorder in accordance with embodiment 3, wherein said misspliced transcripts comprise a polyA 3′ adjacent to said intron sequence.
5. A polynucleotide for use in the treatment of a repeat expansion disorder in accordance with any one of embodiments 1-4, wherein the reduction of said misspliced transcripts is in the cytoplasm.
6. A polynucleotide for use in the treatment of a repeat expansion disorder in accordance with any one of embodiments 1-5, wherein said polynucleotide is complementary to said misspliced transcript.
7. A polynucleotide for use in the treatment of a repeat expansion disorder in accordance with any one of embodiments 1-6, wherein said polynucleotide is complementary to said misspliced transcripts and said complementarity is 5′ from the repeat expansion.
8. A polynucleotide for use in the treatment of a repeat expansion disorder in accordance with any one of embodiments 1-7, wherein said polynucleotide is comprised in a double stranded polynucleotide, said double stranded polynucleotide capable of inducing RNA interference.
9. A polynucleotide for use in the treatment of a repeat expansion disorder in accordance with any one of embodiments 1-8, wherein said misspliced transcript encodes a truncated polyQ protein and wherein said polynucleotide induces a reduction of said truncated polyQ protein.
10. A polynucleotide for use in the treatment of a repeat expansion disorder in accordance with any one of embodiments 3-9, wherein said repeat expansion disorder is Huntington's Disease, said polynucleotide inducing a reduction of misspliced HTT transcripts, wherein the exon with the CAG repeat expansion is exon 1 of HTT and the intron sequence which is 3′ and adjacent therefrom is from intron 1 of HTT.
11. A polynucleotide for use in the treatment of a CAG repeat disorder in accordance with embodiment 10, wherein said misspliced HTT transcripts encode a truncated polyQ HTT protein and wherein said polynucleotide induces in a reduction of said truncated polyQ HTT protein.
12. A polynucleotide for use in the treatment of a CAG repeat disorder in accordance with embodiment 10, wherein said misspliced HTT transcripts encode a truncated polyQ HTT protein and wherein said polynucleotide induces in a reduction of said truncated polyQ HTT protein, wherein said truncated HTT protein comprises an amino acid sequence at its C-terminus of:

(SEQ ID NO. 51)
PPPPPPPPPPPQLPQPPPQAQPLLPQPQPPPPPPPPPPGPAVAEEPLHRP.

13. A polynucleotide in accordance with any one of embodiments 10-12, wherein said polynucleotide is complementary to said misspliced HTT transcript and wherein said polynucleotide is complementary to a sequence selected from

(SEQ ID NO. 5)
5′-GAGACCGCCAUGGCGACCCUGGA-3′;
(SEQ ID NO. 6)
5′-AGACCGCCAUGGCGACCCUGGAA-3′;
(SEQ ID NO. 7)
5′-GAUGAAGGCCUUCGAGUCCCUCAA-3′;
and
(SEQ ID NO. 8)
5′-GGCCUUCGAGUCCCUCAAGUCCUU-3′.

14. A polynucleotide in accordance with any one of embodiments 10-12, wherein said polynucleotide is 5′-AAGGACUUGAGGGACUCGAAGA-3′ (SEQ ID NO. 9).
15. A polynucleotide for use in the treatment of a repeat expansion disorder in accordance with any one of embodiments 3-8, wherein said repeat expansion disorder is SCA3, said polynucleotide inducing a reduction of misspliced ataxin-3 transcripts, wherein the exon with the CAG repeat expansion is exon 10 of ataxin-3 and the intron sequence which is 3′ and adjacent therefrom is from intron 10 of ataxin-3.
16. A polynucleotide for use in the treatment of a CAG repeat disorder in accordance with embodiment 15, wherein said misspliced ataxin-3 transcripts encode a polyQ ataxin-3 protein and wherein said polynucleotide induces in a reduction of said polyQ ataxin-3 protein.
17. A polynucleotide for use in the treatment of a CAG repeat disorder in accordance with embodiment 16, wherein said misspliced ataxin-3 transcripts encode a polyQ ataxin-3 protein comprising an amino acid sequence at its C-terminus of:

(SEQ ID NO. 16)
GDLSGQSSHPCERPATSSGALGSDLGKACSPFIMFATFTLYLTYELHVIFA
LHYSSFPL.

18. A polynucleotide in accordance with any one of embodiments 15-17, wherein said polynucleotide is complementary to said misspliced ataxin-3 transcript and wherein said polynucleotide is complementary to a target sequence within said misspliced ataxin-3 transcript selected from

(SEQ ID NO. 17)
5′-AACACUGGUUUACAGUUAGAAA-3′;
(SEQ ID NO. 18)
5′-AAUUAGGAAAACAGUGGUUUAA-3′;
(SEQ ID NO. 19)
5′-AAGUAUGCAAGGUAGUUCCAGA-3′;
(SEQ ID NO. 20)
5′-UACUUCAGAAGAGCUUCGGAAG-3′;
and
(SEQ ID NO. 21)
5′-GAGACGAGAAGCCUACUUUGAA-3′.

19. A polynucleotide in accordance with any one of embodiments 15-17, wherein said polynucleotide is complementary to said misspliced ataxin-3 transcript and wherein said polynucleotide is selected from:

(SEQ ID NO. 22)
5′-UUUCUAACUGUAAACCAGUGUU-3′;
(SEQ ID NO. 23)
5′-UUAAACCACUGUUUUCCUAAUU-3′;
(SEQ ID NO. 24)
5′-UCUGGAACUACCUUGCAUACUU-3′;
(SEQ ID NO. 25)
5′-CUUCCGAAGCUCUUCUGAAGUA-3′;
and
(SEQ ID NO. 26)
5′-UUCAAAGUAGGCUUCUCGUCUC-3′.

20. A gene delivery vector encoding a polynucleotide in accordance with any one of embodiments 1-9, for use in the treatment of a repeat expansion disorder.
21. A gene delivery vector encoding a polynucleotide in accordance with any one of embodiments 10-14, for use in the treatment of Huntington disease.
22. A gene delivery vector encoding a polynucleotide in accordance with any one of embodiments 15-19, for use in the treatment of SCA3.
23. A gene delivery vector for use in accordance with any one of embodiments 20-22, wherein said gene delivery vector is an AAV gene delivery vector.
24. A gene delivery vector for use in accordance with embodiment 23, wherein said gene delivery vector is an AAV gene delivery vector of serotype 5.
25. A gene delivery vector for use in accordance with any one of embodiments 20-24, wherein said polynucleotide is comprised in a miRNA scaffold.
26. A gene delivery vector for use in accordance with embodiment 25, wherein said miRNA scaffold is a miR451 scaffold.

### EXAMPLES

### Introduction Huntington Disease (HD) Experiments

HD is an inherited, genetic, neurodegenerative disorder that manifests in adulthood with personality changes, movement disturbances and cognitive decline. HD is caused by an expansion of CAG trinucleotide repeats in the exon 1 in the huntingtin gene (HTT) located in chromosome 4 in humans. This mutation results in the translation of a toxic mutant polyglutamine (polyQ)-protein which aggregates and accumulates in the cells. It has been shown that an aberrant splicing of the HTT gene generates a short Exon 1 HTT mRNA transcript with a stop codon one nucleotide in the beginning of intron 1 and a cryptic poly adenylation (polyA) signal more downstream intron 1, which is translated into a pathogenic Exon 1 protein with a polyglutamine tract (Sathasivam et al. 2013). This mis-splicing event has been found in HD mice models and HD patients (Neueder et al. 2017b, 2018). Both full-length HTT and mis-spliced Exon 1 HTT proteins carrying a polyQ tract may form aggregates in the cells and have been involved in HD pathogenesis (FIGS. 1 and 2). However, the expression of the short Exon 1 HTT protein in animal models was sufficient to induce HD phenotypes. Herein, the presence and lowering of the mis-spliced Exon1 HTT mRNA and the pathogenic Exon1 HTT protein by using an AAV-miHTT targeting Exon1 sequence was investigated. For this purpose, two different knock-in HD mice models, and HD patient fibroblast and iPSC-derived neurons will be used in this example.

### Experimental Outline

### Expression Cassettes, miRNAs and AAV Vectors

Expression cassettes and AAV vectors used in the studies are as described i.a. in WO2016102664 and Miniarikova et al., 2016. The expression cassette was inserted into an AAV vector genome backbone flanked by two intact non-coding inverted terminal repeats (ITR) that originate from AAV2. Briefly, miRNA expression cassettes comprise the chimeric chicken-beta actin promoter, the miRNA sequence was replaced by a sequence designed to target a selected gene sequence and engineered in the pri-mir-451 backbone, and the human growth hormone polyA signal. The 22 nucleotide sequence encoding the polynucleotide targeting the Huntington gene sequence, i.e. being fully complementary therewith that was used in these experiments corresponds with 5′-AAGGACTTGAGGGACTCGAAGA-3′ (SEQ ID NO. 50). The sequence targeting the Huntington gene sequence corresponds with the H12 candidate as described in WO2016102664 and Miniarikova et al., 2016, which is incorporated herein by reference. The sequences selected targeting HTT genes represent sequences that, when expressed, and processed by the RNAi machinery, is complementary to target sequences in mRNAs, expressed from mutant HTT genes, in s respectively. Hence, the RNA sequence that is complementary to HTT, when comprised in a miRNA scaffold, corresponds respectively with 5′-AAGGACUUGAGGGACUCGAAGA-3′ (SEQ ID NO. 9). This sequence corresponding with a polynucleotide in accordance with the invention capable of reducing misspliced Htt transcripts, e.g. expressed in a cell and processed by the RNA interference machinery. AAV vectors used in these studies were based on the AAV5 serotype and manufactured using insect cell-based manufacturing. Briefly, Recombinant AAV5 harbouring the expression cassettes were produced by infecting SF+ insect cells (Protein Sciences Corporation, Meriden, Conn., USA) as described (Lubelski et al. Bioprocessing Journal, 2015). Following standard protein purification procedures on a fast protein liquid chromatography system (AKTA Explorer, GE Healthcare, Chicago, Ill., USA) using AVB sepharose (GE Healthcare, Chicago, Ill., USA), the titer of the purified AAV was determined using qPCR.

### HD Mouse Models

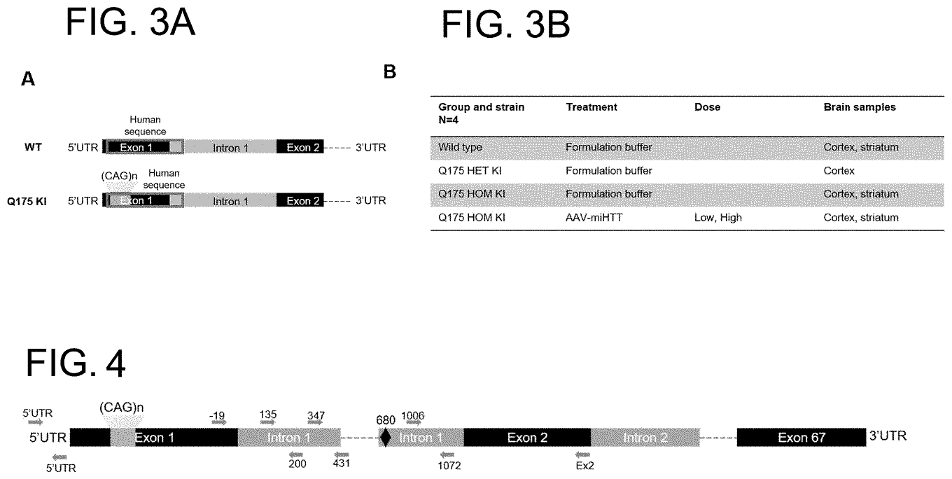

Mouse models used in these experiments include wildtype (WT), Q175KI (Q175 HET KI) (Menalled et all. 2012. PLoS One. 2012; 7(12) and Q175FDN (Q175 HOM KI) (Southwell et al. 2016. Hum Mol Genet. 2016 Sep. 1; 25(17):3654-3675)). Both models have inserted the human exon 1 sequence with 175 CAG repeats (FIG. 3A). Animal groups and brain areas selected are outlined in FIG. 3B.

### Animal Surgery and Tissue Collection

Q175 HET KI mice were bilaterally injected into the striatum at 3 months of age and followed until sacrifice after 12 months post-injection (Q175 HET KI study 1) or at 5 months of age and sacrificed 2 months post-injection (Q175 HET KI study 3) (FIG. 2). The non-treated mice were injected with formulation buffer (PBS+5% Sucrose), treated mice were injected with AAV-miHTT with low dose (5.2×10⁹ genome copies/mouse) or high dose (1.3×10¹¹ genomes copies/mouse). Briefly, mice were anesthetized with 5% isoflurane and placed in a stereotactic frame. During surgery, the concentration of anesthetic was reduced to 1.0%-1.5%. 2-4 μL of vehicle or AAV-miHTT was injected into the striatum in both hemispheres (anterior-posterior [AP], +0.8 mm; medial-lateral [ML], ±1.8 mm; dorsal-ventral [DV], 3.0 mm) using a 10-mL Hamilton syringe at a rate of 0.4 mL/min. The needle was left in place for 3 min after surgery, retracted by 1 mm, and left for another 3 min. Mice were administered buprenorphine (Temgesic, 0.03 mg/kg, 1 mL/kg subcutaneously) one hour before and for 48 h after surgery for analgesia.

Wild type mice and Q175 HOM KI were treated at 3 months of age by stereotaxic bilateral intrastriatal injection under inhalation anaesthesia, followed by 3 months observation period before sacrifice. All injections were performed under aseptic surgical procedures. Q175 HOM KI mice were divided in three groups: non-treated and treated with two different doses. Mice were injected with 2-4 μl of treatment per site. The non-treated mice were injected with formulation buffer (PBS+5% Sucrose), treated mice were injected with AAV-miHTT with low dose (5.2×10⁹ genome copies/mouse) or high dose (1.3×10¹¹ genomes copies/mouse). The WT were only treated with formulation buffer. After treatment, mice were maintained at room temperature on a normal light cycle. They had free access to chow (Lab Diet) and drinking water provided through the cage rack system. After 3 months of treatment, at 6 months of age, mice were sacrificed by Avertin overdose. Mouse brains were extracted immediately following euthanasia and micro-dissected on ice. Both hemispheres of cortex and striatum were collected and then stored at −80° C. until analysis.

### RNA Isolation from Mice

Tissue was crushed using the CryoPrep system (Covaris, Woburn, Mass., USA), and the powder was divided for DNA and RNA analysis. Total RNA was isolated from crushed tissue using a Direct-zol™ RNA MiniPrep kit (Zymo Research).

### Reverse Transcription (RT), PCR and Quantitative RT-PCR

The DNase treatment and the reverse transcription were performed using the Maxima First Strand cDNA Synthesis Kit (Thermo Fisher, K1671). A total of 200 ng total RNA was treated with 1 μl of 10× dsDNase Buffer and 1 μl of dsDNase and incubated for 5 min at 37° C. Then, RNA was reverse transcribed with 4 μl of 5× Reaction Mix, 2 μl of Maxima enzyme Mix and 4 μl of free-RNAse water. After the RT reaction, the cDNA was diluted 1:10 in water.

All PCRs were carried out using the Platinum Green Hot system (Platinum™ Green Hot Start PCR Master Mix (2×), Invitrogen™, 13001012). Each PCR contained 5 μL of 2× Platinum, 2 μL of 5× GC enhancer, each 0.2 μL of 1000 μM primers, 2 μL cDNA template and water to 10 μL. PCR protocols was as follow: 1 cycle 98° C. for 3 min, 35 cycles 98° C. for 15 sec, 59° C. for 20 sec, 72° C. for 30 sec, 1 cycle 72° C. for 5 min. The product was migrated in 2% Agarose Gel contained SYBR™ Safe DNA Gel Stain (6 μL/100 mL of buffer, Invitrogen™) diluted in 0.5 TAE Buffer (Tris base, acetic acid, EDTA) for 2 h at 75V. Bands were excised from the gel, the DNA was purified with the GeneJET Gel Extraction Kit (Thermo Fisher™, K0691), and sequenced by BaseClear B.V. (Leiden). Primer set were Exon 1-Intron 1: −19f/431r, Exon 2: ex2f/ex2r, Exon 1-exon 2: −19f/Ex2r, Intron 1: 347f/785r and Intron 3: int3f/int3r.

Quantitative RT-PCR (qRT-PCR) were performed using TaqMan Universal Master Mix II (Thermo Fisher™, 4440044). For all the primer-probe sets, the concentration was 30 uM primers+6 uM probe (based on EXP19000229). Each RT-qPCR contained 5 μL of TaqMan Universal Master Mix II with UNG, 0.5 ul of primer-probe mix (0.15 μL of each 100 μM primers, 0.03 μL of 100 μM probes), 4 μL of cDNA template and water to 10 μL. qPCR protocol was a follow: 1 cycle 95° C. for 20 sec, 40 cycles 95° C. for 3 sec, 60° C. for 30 sec. Results were normalized to expression level of housekeeping genes (GAPDH, Atp5b, Ubc) with 7500 Software v2.3. Expression levels were quantified by Pfaffl's method, calculating the GeoMean of 3 HK genes and assuming a probe efficiency (E) equal to 2.

$$Relative gene expression = \frac{{( {E}_{GOI} )}^{Δ Ct GOI}}{GeoMean [ {( {E}_{REF} )}^{Δ Ct REF} ]}$$

Primers were designed to bind to specific sequences of the mouse HTT mRNA. All primers used are listed in Table 1 and 2. The set of primers for qPCR are listed in Table 3 and for PCR in Table 4. Scheme of the mouse HTT gene and location of primers is showed in FIG. 4.

TABLE 1
Primers and probes sequences for mouse model.
All these primers were purchased in Eurofins Genomics
and probes in Applied Biosystems UK.

| | | | Position | | | |
|---|---|---|---|---|---|---|
| | SEQ | | start/end in | | | |
| | ID | | bp (start from | Size | | Tm |
| Name | NO. | Sequence (5′ to 3′) | HTT Exon 1) | (nt) | Function | (° C.) |
| -19 fw | 27 | AGGAACCGCTGC | 348-364 | 17 | PCR, qPCR | 57.6 |
| | | ACCGA | | | | |
| 431 rv | 28 | GAGACCTCCTAA | 771-797 | 27 | PCR, qPCR | 61.9 |
| | | AAGCATTATGTC | | | | |
| | | ATC | | | | |
| Ex2 fw | 29 | AAGAAGGAACTC | 21000-21021 | 22 | PCR, qPCR | 60.3 |
| | | TCAGCCACCA | | | | |
| Ex2 rv | 30 | CTGAGAGACTGT | 21060-21082 | 23 | PCR, qPCR | 60.6 |
| | | GCCACAATGTT | | | | |
| 347 fw | 31 | TCCTCATCAGGC | 713-735 | 23 | PCR, qPCR | 64.2 |
| | | CTAAGAGCTGG | | | | |
| 785 rv | 32 | TGAAAACTGAGC | 1130-1151 | 22 | PCR | 58.4 |
| | | ACCACCAATG | | | | |
| 5′UTR | 33 | CTTGGTTCCGCTT | 44-61 | 18 | qPCR | 58.2 |
| fw | | CTGCC | | | | |
| 5′UTR | 34 | TGGAGCCTACTG | 107-126 | 20 | qPCR | 63 |
| rv | | GCACTACG | | | | |
| 5′UTR | 35 | CAGAGCCCCATT | 69-92 | 24 | qPCR | |
| p | | CATTGCCTTGCT | | | | |
| 135 fw | 36 | CTTGCGGGGTCT | 501-517 | 17 | qPCR | 60 |
| | | CTGGC | | | | |
| 200 rv | 37 | TCAGCGAGTCCC | 549-566 | 18 | qPCR | 60.5 |
| | | TGGCTG | | | | |
| 155 p | 38 | CCTCAGAGGAGA | 521-545 | 25 | qPCR | |
| | | CAGAGCCGGGTC | | | | |
| | | A | | | | |
| 431 rv | 39 | GAGACCTCCTAA | 771-797 | 27 | qPCR | 61.9 |
| | | AAGCATTATGTC | | | | |
| | | ATC | | | | |
| 371 p | 40 | AGTGCAGGACAG | 737-761 | 25 | qPCR | |
| | | CGTGAGAGATGT | | | | |
| | | G | | | | |
| Ex2 p | 41 | AGAAAGACCGTG | 21022-21058 | 37 | qPCR | |
| | | TGAATCATTGTC | | | | |
| | | TAACAATATGTG | | | | |
| | | A | | | | |

Fw: forward, rv: reverse, p: probe, Tm: temperature melting.

TABLE 2
TaqMan Gene expression assays for mouse model. These assays were
purchased in Applied Biosystems by Thermo Fisher Scientific.

| Gene targeted | Number |
|---|---|
| HTT Exon 64-65 | Mm01213820_m1 |
| GAPDH | Mm99999915_g1 |
| Atp5b | Mm01160389_g1 |
| Ubc | Mm01198158_m1 |

TABLE 3
Set of specific primers used for the qPCR for mice KI model.

| Region | Primers |
|---|---|
| 5′UTR | 5′UTR fw/5′UTR rv/5′UTR p |
| Exon 1-2 | −19 fw/Ex2 rv/Ex2 p |
| Exon 2 | Ex2 fw/Ex2 rv/Ex2 p |
| Early intron 1 | 135 fw/200 rv/155 p |
| Intron 1 | 347 fw/431 rv/371 p |
| Human Exon1-intron1 | −17 fw/Ex2 rv/Ex2 p |

Fw: forward, rv: reverse, p: probe.

TABLE 4
Set of specific primers used for the PCR for mice KI model.

| Region | Primers |
|---|---|
| Exon 1-2 | −19 fw/Ex2 rv |
| Exon 1-Intron 1 | −19 fw/431 rv |

Fw: forward, rv: reverse, p: probe.

In FIG. 4, a schematic outlining the location of primers used is presented.

### OligodT Reverse Transcription and 3′RACE

The DNAse treatment was performed as described above. Then the RNA was reverse transcribed with anchored-oligonucleotide(dT)-tailed primer (QT 3′RACE for mouse or QT primer for human, Integrated DNA Technologies). A total of 200 ng of total RNA was treated with 4 μL of 5× Reverse Transcription Buffer, 1 μL of 10 mM dNTP solution (Thermo Fisher™), 2 μL of 0.1 M DTT, 0.5 μL of 100 ng/μL QT primer, 0.25 μL of 40 U/μL RNasin (RNasin® Ribonuclease Inhibitors Plus, Promega), 200 U of Superscript IV RT (SuperScript™ IV Reverse Transcriptase kit, Invitrogen™) and water up to 20 μL. The reaction mix was treated as follows: 1 h at 42° C., 10 min at 50° C. and 15 min at 70° C. Then the cDNA was digested with 1.5 U of RNAseH (Thermo Fisher™) and incubated for 20 min at 37° C.

The Rapid Amplification of cDNA Ends (3′RACE) was performed according to Sathasivam et al, 2013. Each 3′RACE consisted of 2 rounds of amplification by PCR with gene-specific primers mentioned below (Table Y) Each 3′RACE contained 1 ul of non-diluted cDNA, 5 μL of 5× buffer, 2 μL of 25 mM MgCL₂ solution, 0.5 μL of 10 mM dNTP solution, each 0.05 μL of 100 μM primers, 0.125 μL of GoTaq and water to 25 μL. Primers used for the first round were Q₀ and 571 fw, the uncolored 5× buffer and the program as follow: 1 cycle for 2 min at 94° C., 10 cycles for 15 sec at 94° C., 25 sec at 59° C., 2 min at 72° C., 30 cycles for 15 sec at 94° C., 20 sec at 59° C., 1 min 45 sec at 72° C., 1 cycle for 6 min at 72° C. Second round was performed with Qᵢ and 622 fw primers, the green 5× buffer and as follow: 1 cycle for 2 min at 94° C., 35 cycles for 15 sec at 94° C., 20 sec at 62° C., 1 min at 72° C., 1 cycle for 6 min at 72° C.

TABLE 5
Primers sequences for 3′RACE mouse model.
All these primers were purchased in Eurofins Genomics.

| | | | Position | | |
|---|---|---|---|---|---|
| | SEQ | | start/end in bp | | |
| | ID | | (start from | Size | Tm |
| Name | NO. | Sequence | HTT Intron 1) | (nucleotides) | (° C) |
| Q_{T} | 42 | CCAGTGAGCAGAGTGACGAG | polyA tails | 52 | 66.9 |
| 3′RACE | | GACTCGAGCTCAAGCTTTTTT | | | |
| | | TTTTTTTTTTT | | | |
| Q₀ | 43 | CCAGTGAGCAGAGTGACG | / | 18 | 58 |
| Qᵢ | 44 | GAGGACTCGAGCTCAAGC | / | 18 | 58 |
| 571 fw | 45 | AACCAGGTTTTAAGCATAGCC | 571-594 | 24 | 59 |
| | | AGA | | | |
| 622 fw | 46 | AGTTGGATGAGTTGTATTTGT | 622-651 | 30 | 61 |
| | | CAAGTACAT | | | |

fw: forward, Tm: temperature melting.

Detection of Biodistribution and Exon 1 HTT mRNA in Heterozygote Q175KI Mice 2 Months After Intrastriatal Injection of AAV5-miHTT

All animal experiments were performed as specified in the license authorized by the national Animal Experiment Board of Finland and according to the National Institutes of Health (Bethesda, Md., USA) guidelines for the care and use of laboratory animals. 30 (15 females and 15 males) heterozygote Q175 KI mice (Menalled et all. 2012. PLoS One. 2012; 7(12)) and 10 wild-type (WT) (5 females and 5 males) littermate mice obtained from Charles River Germany (Sulzfeld, Germany) were treated at 5 months of age. Bilateral intra-striatal injection of vehicle or AAV5-miHTT (low or high dose) was administered by using sterotactically guided Hamilton syringes and infusion system (Harvard Apparatus). Mice were sacrificed at 8 weeks post-treatment (7 months of age). After perfusion with heparinized saline the brain are organs were collected. Both brain hemispheres were dissected into striatum, two samples from cortex (frontal and caudal parts, same cortical areas for all animals), hippocampus, thalamus, cerebellum and the rest of brain. The dissected pieces were weighed pre-cooled round bottom safe lock 2 ml Eppendorf tubes and frozen on dry ice and stored at −80° C. The spinal cord was collected as whole, and cut in three equally long pieces, each placed in a 2 ml Eppendorf tube and frozen on dry ice and stored at −80° C. In addition, one lobe of liver was collected and frozen on dry ice and stored at −80° C.

To investigate the biodistribution of miHTT and target engagement of exon 1 mRNA in the brain, tissue from left frontal cortex and caudal cortex was used to isolate RNA by using Direct-zol™ RNA MiniPrep kit (Zymo Research). To determine miRNA expression levels, two-step RT-qPCR was performed by TaqMan Fast Universal kit (Thermo Scientific, MA, USA), and custom stem-loop primer/probe for detection of miHTT. Expression levels of miHTT were calculated based on a standard line with synthetic RNA oligos (Integrated DNA Technologies, IA, USA).

To determine the presence and lowering of exon 1 HTT mRNA and full-length HTT mRNA, two-step RT-qPCR was performed. First, DNase treatment and reverse transcription were performed using the Maxima First Strand cDNA Synthesis Kit (Thermo Fisher, K1671). A total of 200 ng total RNA was treated with 1 μl of 10× dsDNase Buffer and 1 μl of dsDNase and incubated for 2 min at 37° C. Then, RNA was reverse transcribed with 4 μl of 5× Reaction Mix, 2 μl of Maxima enzyme Mix and 4 μl of free-RNAse water. After the RT reaction, the cDNA was diluted 1:5 in water for injection.

Quantitative RT-PCR (qRT-PCR) were performed using TaqMan Fast Universal Master Mix (Thermo Fisher™). For all the primer-probe sets, the concentration was 30 uM primers+6 uM probe. Each RT-qPCR contained 5 μL of TaqMan Fast MasterMix 0.5 ul of primer-probe mix (0.15 μL of each 100 μM primers, 0.03 μL of 100 μM probes), 4 μL of cDNA template and water to 10 μL. qPCR protocol was a follow: 1 cycle 95° C. for 20 sec, 40 cycles 95° C. for 3 sec, 60° C. for 30 sec.

Primers were designed to bind to specific sequences of the mouse HTT mRNA and human exon 1 sequence. Primer and TaqMan probes combinations used are listed in Table 2. Sequences of primers are previously listed in Table 1. Scheme of the mouse HTT gene and location of primers is showed in FIG. 4.

Results were normalized to the geometric mean of the expression level of three housekeeping (HK) genes (GAPDH, HPRT and PGK1). Expression levels were quantified by Pfaffl's method, calculating the GeoMean of three HK genes and assuming a probe efficiency (E) equal to 2.

### Determination of HTT Protein Variants in Mouse Cortical Tissues

Protein analysis was performed by Homogeneous Time Resolved Fluorescence (HTRF). For this, tissue samples from right hemisphere were weighted and lysed at a 10% concentration in 1% Triton in PBS+Protease inhibitors. Combination of specific antibodies was used to detect the different HTT protein specifies

| HTT protein species | Donor antibody | Receptor antibody |
|---|---|---|
| Soluble exon 1 HTT protein | 2B7-Tb | MW8-d2 |
| Soluble mutant HTT | 2B7-Tb | 4C9-488 |
| | 4C9-Tb | MW1-d2 |
| Soluble full-length HTT | MAB5490-Tb | MAB2166-d2 |
| (wt and mutant HTT) | | |
| Exon 1 HTT aggregation | 4C9-Tb | MW8-d2 |

Fibroblast and iPSC-derived Neuron Culture

Fibroblasts and iPS cells derived from SCA3 patients, HD patients and controls were purchased. The HD fibroblasts and reprogrammed iPSCs were derived from an HD patient with 180 CAG repeats. Fibroblasts were maintained in MEM medium (Thermo Fisher) supplemented with 2 mM L-Glutamine, 15% Fetal Bovine Serum and 1% Penicillin/Streptomycin. Cells are kept in culture up to 80% confluency in 24-well plate, and then washed with PBS, detached with 300 μl of Trizol and collected in new 1.5 mL tubes for storage at −80° C. until analysis. Total RNA is isolated using the Direct-zol™ RNA MiniPrep kit (Zymo Research).

iPSCs were maintained on matrigel coating with mTeSR medium. For the neural induction, cells were plated onto AggreWell 800 plates at day 0 as 3×106 cells per well in STEMdiff Neural Induction Medium. At day 5, embryoid bodies were formed and replated onto poly-D-lysine/laminin coated 6-well plates. At day 12, the neuronal rosettes were selected using STEMdiff Neural Rosette Selection Reagent and replated in poly-D-lysine/laminin coated plates. Next day, differentiation of neural progenitor cells was initiated using STEMdiff Neuron Differentiation Kit. From day 19, cells were matured using STEMdiff Neuron Maturation Kit for a minimum of two weeks.

Detection and lowering of Exon1 HTT mRNA and protein in HD fibroblast and HD iPS-derived neurons.

HD fibroblast and iPSC-derived neurons both carrying 180 CAG repeats are used to investigate the lowering of full-length and Exon1 HTT mRNA and protein in a human-based in vitro system. Control cells without the CAG repeat expansion are taken along. Briefly, patient-derived cells are incubated with AAV-miHTT, antisense oligonucleotides and/or siRNAs targeting Exon 1 HTT and 3′ of the repeat. Molecular techniques such as 3′RACE, RT-qPCR, immunoprecipitation-Western Blot (IP-WB) and Time-resolved fluorescence energy transfer (TR-FRET) are used to measure Exon 1 HTT transcript and protein. The expected outcome is a dose-dependent lowering of both full-length and short Exon 1 HTT mRNA by oligonucleotides targeting Exon 1 HTT sequences, as opposed to selective lowering of full-length HTT mRNA by oligonucleotides targeting 3′ sequences. In the same manner, the expected outcome is a dose-dependent lowering of full-length and pathogenic Exon1 HTT protein detected by TR-FRET or equivalent methods by oligonucleotides targeting Exon1 sequences as opposed to oligonucleotides targeting 3′ sequences.

Detection and lowering of exon 11 lacking ATXN3 mRNA and protein in SCA3 fibroblast and HD iPS-derived neurons.

SCA3 fibroblast and iPSC-derived neurons will be used to investigate the lowering of full-length and exon 11 lacking ATXN3 mRNA and protein in a human-based in vitro system. Control cells without the CAG repeat expansion are taken along. Briefly, patient-derived cells are incubated with AAV-miATXN3, antisense oligonucleotides and/or siRNAs targeting ATXN3 exon 1 to 10 and 3′ of the repeat. Molecular techniques such as 3′RACE, RT-qPCR, immunoprecipitation-Western Blot (IP-WB) and Time-resolved fluorescence energy transfer (TR-FRET) will be used to measure exon 11 lacking ATXN3 transcript and protein. The expected outcome is a dose-dependent lowering of both full-length and exon 11 lacking ATXN3 mRNA by oligonucleotides targeting exon 11 lacking ATXN3 sequences, as opposed to selective lowering of full-length ATXN3 mRNA by oligonucleotides targeting 3′ sequences. In the same manner, the expected outcome is a dose-dependent lowering of full-length and pathogenic exon 11 lacking ATXN3 protein detected by TR-FRET or equivalent methods by oligonucleotides targeting exon 11 lacking ATXN3 sequences as opposed to oligonucleotides targeting 3′ sequences.

### Detection and Lowering of Exon 1 HTT Protein in Striatum and Cortex of Humanized Mice (Hu128/21)

The presence and lowering of full-length and Exon 1 HTT mRNA and protein in a fully humanized heterozygous mouse model (Hu128/21) (Southwell et al 2017, Hum Mol Genet. 2017, 26(6):1115-1132) after treatment with AAV-miHTT is investigated. Hu128/21 and control Hu21 animals received bilateral intrastriatal injections infusions by convection-enhanced delivery (CED) of either saline or 3 ascending doses of AAV5-miHTT (low: 5.2×10⁹, medium: 2.6×10¹⁰, or high: 1.3×10¹¹ genome copies per mouse) at 2 months of age and were evaluated until 9 months of age. Target engagement of Exon 1 HTT is assessed by quantification of HTT suppression using Western blot at 4 months post AAV5-miHTT injection. Preliminary results indicate that a significant reduction of HTT exon 1 protein with all doses of AAV5-miHTT in both the striatum and cortex at 4 months post-injection is achieved.

Detection of HTT exon 1 fragment in Hu128/21 mouse primary cortical neurons and lowering by AAV5-miHTT.

This study is performed at the Centre for Molecular Medicine and Therapeutics, University of British Columbia. In brief, brains from E16.5 Hu128/21 embryos are removed, cortices micro-dissected, dissociated to single cells and seeded into 6 well plates at a density of 1×10⁵ cells/cm2 (approximately 1×10⁶ cells/well). At day 5 of maturation, cells are treated with AAV5-miHTT (targeting exon 1) and AAV-miSNP50 (targeting exon 50) at 3 doses, multiplicity of infection (MOI) 1×10⁵, 1×10⁶, 1×10⁷ (gc/cells). As a control, cells are treated with vehicle or AAV5-GFP. Each treatment is performed in triplicate and each experiment is replicated. Following a treatment duration of 10 days, morphology and viability of cells is assessed qualitatively prior to harvest. Cell pellets are freeze at −80 C. Target engagement of Exon 1 HTT is assessed by quantification of HTT suppression using HTRF with different antibodies for the detection of exon 1 HTT protein and full-length HTT protein. The expected outcome is a significant dose-dependent reduction of HTT exon 1 protein and full-length HTT protein with AAV5-miHTT targeting exon 1. On the contrary, the expected outcome of the treatment of cells with AAV5-miSNP 50 targeting exon 50 is a significant lowering of full-length HTT protein, but a non-significant reduction of exon 1 HTT protein. The expected outcomes confirm that AAV-delivered miRNA therapeutics targeting exon 1 HTT sequence result in lowering of pathogenic exon 1 HTT protein, as opposed to other therapeutics targeting sequences downstream exon 1 sequence.

### Detection and Lowering of Exon 1 HTT Aberrant Transcript in Biofluids (Plasma and Cerebrospinal Fluid) of Huntington Disease Patients

The presence of Exon 1 HTT mRNA in extracellular vesicles isolated from plasma and/or cerebrospinal fluid samples from healthy volunteers and Huntington disease patients is investigated. Biofluid samples (obtained for research purposes with informed consent) from

Huntington disease patients treated with AAV-miHTT are used to evaluate the effects of treatment on Exon1 HTT mRNA levels.

### Detection and Lowering of Exon 1 HTT Protein in Biofluids (Plasma and Cerebrospinal Fluid) of Huntington Disease Patients

Specific and sensitive antibody-based assays are developed to investigate the presence of Exon1 HTT protein in plasma and/or cerebrospinal fluid samples from healthy volunteers and Huntington disease patients. Biofluid samples (obtained for research purposes with informed consent) from Huntington disease patients treated with AAV-miHTT will be used to evaluate the effects of treatment on Exon1 HTT protein levels.

### Results

Successful Detection of Aberrantly Spliced Exon1 HTT mRNA in Q175 KI HET and HOM HD Mouse Models

In order to investigate the presence of the Exon 1 HTT mRNA transcript in the Q175 KI HET and HOM HD mouse models, we used several techniques that allow for the qualitative and quantitative detection of Exon 1 HTT mRNA. Specific primers were designed to target different parts of the HTT mRNA (FIG. 4). and selectively measure the expression of the mis-spliced Exon 1 HTT mRNA or the full-length HTT mRNA.

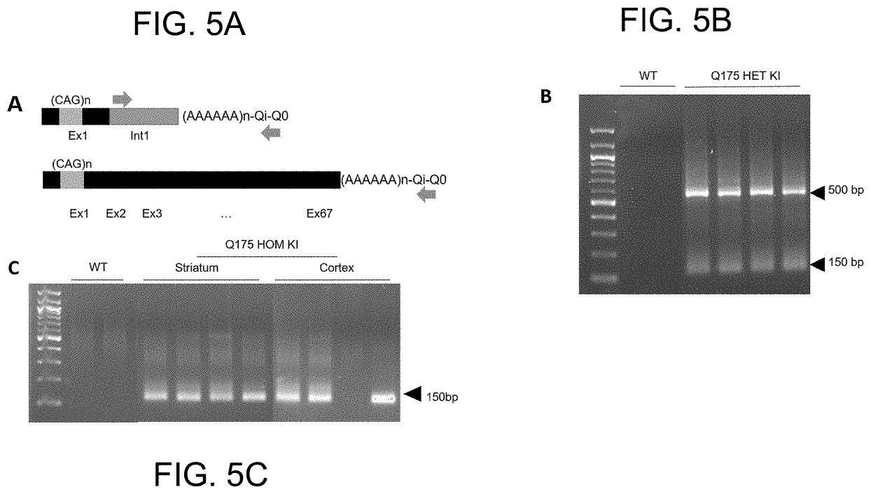

First, 3′RACE was performed to qualitatively detect the presence of the mature polyadenylated HTT Exon 1 mRNA. For this, we used an anchored oligonucleotide(dT)-tailed primer in the RT step. Then specific primers targeting HTT Intron 1 sequences and internal sequence (Q0 and Qi) of the oligodT primer were used to amplify the polyadenylated HTT Exon 1 mRNA transcript by 2 rounds of PCR (FIG. 5A). This method allows us to specifically detect the presence of the mis-spliced mature HTT Exon 1 mRNA. As expected from previous studies, HTT Exon 1 mRNA was not present in the WT mice (FIG. 5B and FIG. 5C). In Q175 KI HET mice, two products were detected of 500 bp and 150 bp. (FIG. 5B). The detection of these two different products is due to the presence of two cryptic polyA sites in HTT Intron 1 in HD mice (as described in Sathasivam et al. 2013), one at 680 bp and the second one at 1145 bp (1.2 kb site). The bands were extracted from the agarose gel and sequenced by Sanger sequencing, which confirmed that the products correspond to two Intron1-containing products.

Likewise, we detected a short polyA mRNA transcript containing Intron1 sequence in the Q175 KI HOM HD mouse model, but not in WT mice from the same background strain (FIG. 5C). This Intron 1-containing transcript was detected in both the striatum and the cortex as a single band around 150 bp (FIG. 5C). Sanger sequencing was used to confirm the Intron 1 sequence.

Confirmed sequence by Sanger sequencing:

Medium transcript (corresponds to Mus musculus Intron1 up to cryptic polyA site at 1.2 kb)

(SEQ ID NO. 47)
CGTNNNATTTCTTAGGTGTGATTATTAATAAAAAACTATATGTGTGCATAT
ATATGAAAGAGTCGACTTATACTTAACTGCCTATCGATTTTTTGTTCTATA
TAAAACGGATACATTGGTGGTGCTCAGTTTTCACCGGGGAATGAATTTTAC
TAGTGTTGCAGACAGGCTTGTTTTAGAACATAGGCCACTCTGACTCTGACT
TTGTGCCAGTAAAAGTTCCTGTTTAGTTCTTTGCTGACATCTTATAGATCT
TTGGAAGCTAGCTGCTTGTGACTGGAGAGAATATTGAAACANAAGAGAGAC
CATGAGTCACAGTGCTCTAAGAGAAAAGAGACGCTCAAAACATTTCCTGGA
AATCCATGCTGAGTGTTGAGCCCTGNGCTCTCTTGCAGCTCAGTCCTTTCT
CTCAACTCTGGGCATTTTATTTCTAATCTGGATTTGTATAATTAATAAGGA
GAACTTTTGGGAACAACCTACTAAAGAATGTCATCATTAAAACTCATTANA
ATC

Short transcript (too short for sequencing, corresponds to Mus musculus Intron 1 up to cryptic polyA site at 680 bp)

(SEQ ID NO. 48)
GGTGNANTNTATTANGTGTGATTATTATAAAAAACTATATGTGTGCATATA
AAAAAAAAAAAAAAAAAAAAAAAAAAAA.

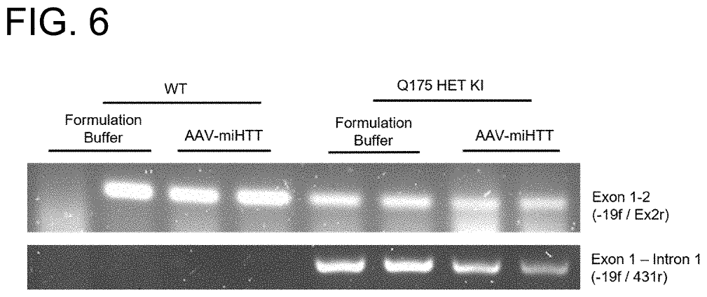

In order to confirm these results, we performed a PCR using primers targeting Exon 1-Intron 1 sequence. Particularly, two sets of primers were used for RT-PCR analysis: one to detect the full-length HTT mRNA (“Exon 1-2”, −19fw and Ex2rv primers) and the other one to detect the mis-spliced Exon 1 HTT mRNA (“Exon 1-Intron 1”, −19fw and 431rv primers) (FIG. 6). Expectedly, the “Exon 1-Intron 1” sequence was not detected in the WT mice because the intron 1 sequence is spliced by the spliceosome into mature mRNA. Differently, we detected a polyadenylated “Exon 1-Intron 1” sequence in the cortex Q175 HET KI mice (FIG. 6). Sanger sequencing of the end-product was used to confirm the Exon1-Intron1 sequence. Furthermore, the “Exon 1-2” sequence, corresponding to the full-length HTT mRNA, was present in both WT and Q175 HET KI mice. However, the bands for “Exon 1-2” sequence are less intense in Q175 HET KI, likely because a fraction of the full-length mRNA undergoes aberrant splicing resulting into a mature Exon 1 HTT mRNA.

Sequence confirmed by Sanger sequencing: Exon 1-Intron1

(SEQ ID NO. 49)
GCCNCCCNGTGAGCAGGCTTTCCGGCCCGGGCCCTCGTCTTGCGGGGTCTC
TGGCCTCCCTCAGAGGAGACAGAGCCGGGTCAGGCCAGCCAGGGACTCGCT
GAGGGGCGTCACGACTCCAGTGCCTTCGCCGTTCCCAGTTTGCGAAGTTAG
GGAACGAACTTGTTTCTCTCTTCTGGAGAAACTGGGGCGGTGGCGCACATG
ACTGTTGTGAAGAGAACTTGGAGAGGCAGAGATCTCTAGGGTTACCTCCTC
ATCAGGCCTAAGAGCTGGGAGTGCAGGACAGCGTGAGAGATGTGCGGGTAG
TGGATGACATAAT.

In order to further quantify the expression level of the HTT Exon 1 mRNA, a RT-quantitative

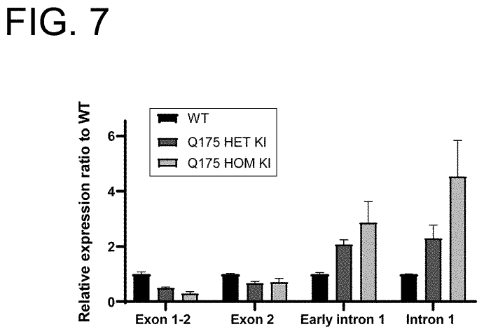

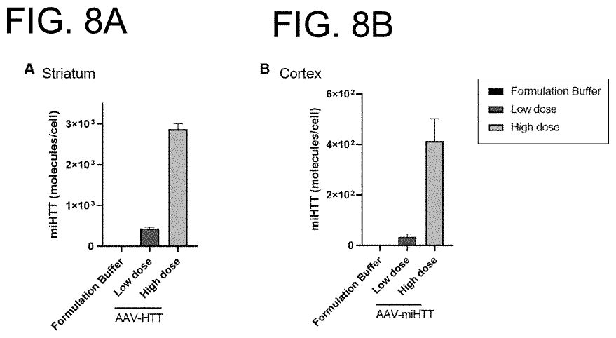

PCR (RT-qPCR) was performed. Four sets of specific primers (“Exon 1-2”, “Exon 2”, “Early intron 1” and “Intron 1”) were used to target different sequences of the HTT mRNA to specifically measure the levels of both full-length and Exon 1 HTT mRNA (FIG. 7). To clarify, the “Exon 1-2” and “Exon 2” primers were used to measure the expression of the full-length HTT mRNA. We detected a lower expression of these two products in both Q175 mice, relative to the WT mice, likely because one fraction of HTT mRNA undergoes alternative splicing and generates Exon1 HTT mRNA in these models (FIGS. 8A and 8B). Moreover, the expression level of the full-length HTT is relatively lower in the Q175 HOM KI mice since both alleles undergo mis-splicing compared to Q175 HET KI mice. However, the expression of the “exon 2” sequence, which also corresponds to the full-length HTT, was not lower in both HD mice models when compared to WT mice. This could be explained by different primer efficiency between primer sets. In order to measure the expression level of the mis-spliced HTT Exon 1 transcript, different sequences of the Intron 1 were measured by TaqMan qPCR (“early intron 1” and “intron 1”). Both primer sets target sequences upstream of the first polyA cryptic site (680 bp). In both Q175 KI mice models, the “Early intron 1” and “Intron 1” primers showed a higher expression compared to WT mice (FIG. 7). The difference in expression level between early intron 1 and intron 1 could be due to the primer efficiency. The expression of the Intron 1 was up to 4.5 times higher in Q175 KI than in WT mice.

All together, these results obtained by different qualitative and quantitative techniques confirmed the presence and detection of polyadenylated HTT Exon 1 mRNA in two different HD KI mice models due to aberrant splicing.

Dose-dependent Expression of miHTT in Striatum and Cortex of Q175 HOM KI Mice Upon AAV-miHTT Treatment

We first evaluated the expression level of the therapeutic miHTT in different areas of Q175 HOM KI. Mice were intrastriatally injected with two different doses of AAV-miHTT (ns=4 per group). Six months after the injection mice were sacrificed, and striatum and cortex were collected separately. In order to measure the miHTT expression levels in the brain, RT-qPCR was performed on samples from the striatum and the cortex. In both striatum and cortex, we detected dose-dependent levels of miHTT after treatment (FIGS. 8A and FIG. 8B). In the striatum, where AAV-miHTT was infused directly, there was a higher expression of miHTT, up to 7×10³ molecules per cells in mice treated with the high dose (FIG. 8A). In the cortex, we also measured high levels of expression of miHTT, up to 5.5×10² molecules per cell in mice treated with high dose (FIG. 8B). As expected, the level of expression of miHTT in the striatum, site of injection, is higher compared to the cortex.

Lowering of HTT Exon 1 mRNA by AAV-miHTT Treatment in Q175 HOM KI Mice

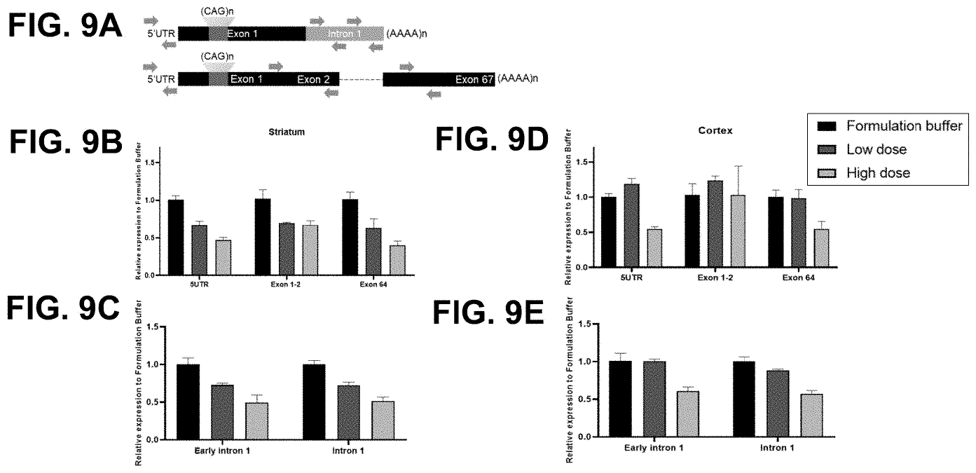

Since AAV-miHTT was designed to target HTT Exon 1, the goal of this project is to determine whether AAV-miHTT treatment can reduce both full-length HTT and Exon 1 HTT mRNAs in HD mice. For this purpose, we quantified the expression level of different HTT sequences by oligo-dT RT-qPCR using different set of primers and probes (5′UTR, Exon 1-2, Exon 64, Early intron 1, Intron 1) (FIGS. 9A-9E). Primers were designed to selectively measure the full-length HTT mRNA or the Exon 1 HTT mRNA expression levels (FIG. 9A). We then compared the expression levels in AAV-miHTT-treated Q175 HOM KI mice with non-treated mice to determine the on-target lowering of full-length and Exon1 HTT mRNA in different brain areas (n=4 per treatment). In the striatum, we observed a dose-dependent lowering of the 5′UTR sequence—which is present in both HTT full-length and Exon 1 mRNAs—when compared to non-treated group (FIG. 9B). Moreover, the expression of Exon 1-2 and Exon 64 sequences was reduced in a dose-dependent manner in the striatum upon AAV-miHTT treatment (FIG. 9B). This means that AAV-miHTT treatment resulted in a significant lowering of the full-length HTT mRNA. Two sequences of the Intron 1 (“early intron 1” and “intron 1”) were measured to selectively investigate the expression level of the mis-spliced Exon 1 HTT mRNA. For both sequences, we observed a dose-dependent lowering of the intron 1 expression upon treatment in the striatum (FIG. 9C). High dose AAV-miHTT treatment resulted in up to 50% lower expression of Exon1 HTT mRNA compared to non-treated.

In the cortex, distal from injection site, we observed a significant lowering of 5′UTR sequence in mice treated with the high dose of AAV-miHTT, but not with the lower dose (FIG. 9D). It is likely that the level of miHTT in the cortex after a low dose intrastriatal injection is not sufficient to induce a significant lowering of HTT mRNA. In the same manner, a significant lowering of the intron 1 sequence was detected in the cortex upon treatment with high dose AAV-miHTT, but not with the low dose treatment (FIG. 9E). All together, these results showed a dose-dependent lowering of both full-length HTT and mis-spliced Exon 1 HTT mRNAs in the striatum and cortex in Q175 HOM KI mice. In other words, we confirmed that AAV-delivered miHTT can target and significantly lower the mis-spliced Exon 1 HTT mRNA in vivo, providing an important therapeutic advantage.

Lowering of Exon 1 HTT mRNA and Protein in Frontal Cortex of Q175 KI HET mice at 2 Months After Intrastriatal Treatment of AAV-miHTT

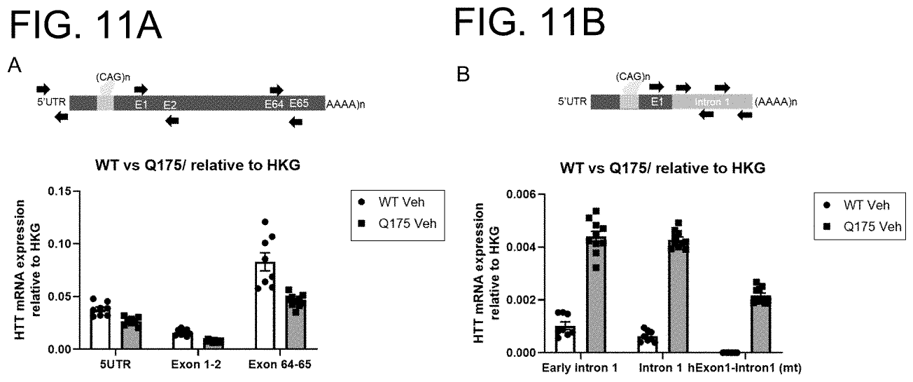

The goal of this study is to investigate the lowering of full-length HTT and Exon 1 HTT mRNA and protein in HD mice after 2 months of intrastriatal AAV-miHTT treatment. For this purpose, the presence and detection of exon 1 HTT mRNA was first evaluated in Q175KI HET mice in comparison to WT mice. The expression level of different HTT sequences was measured by RT-qPCR using different set of primers and probes. Full-length HTT mRNA levels were detected by primer/probe sets “5′UTR”, “Exon 1-2” and “Exon 64-65”, and exon 1 HTT mRNA levels were detected by primer/probe set “Early intron 1”, “Intron 1” and “human exon1-intron1” (specific for mutant exon1 mRNA (FIG. 11). The expression levels in the frontal cortex of Q175KI HET and WT mice was compared to expression levels of three housekeeping genes. Results showed a lower expression of full-length HTT sequences (primer set “5′UTR”, “exon 1-2” and “exon 64-65”) in Q175KI HET compared to WT mice (FIG. 11). Moreover, we detected a higher expression of exon 1 HTT transcript (primer set “early intron 1” and “intron 1”) in Q175KI HET mice compared to WT (FIGS. 11A and 11B). Since exon 1-intron 1 HTT mRNA was not expected to be present in WT mice, low detection levels of intron 1 might be due to DNA contamination or background of the assay. Moreover, quantification of “human exon1-intron1” sequences validated that human exon 1 sequences are only present in Q175KI HET and not in WT mice.

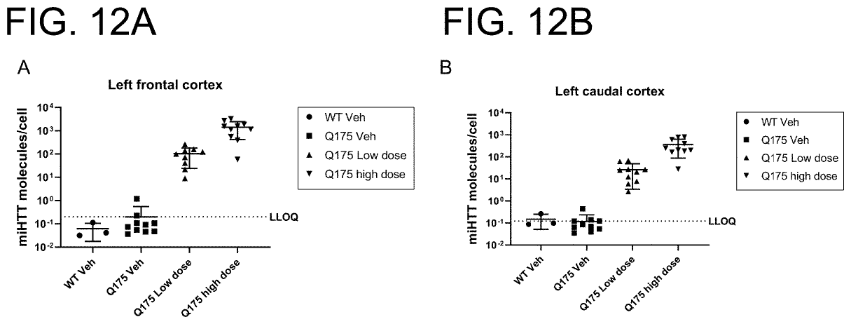

To investigate the biodistribution of AAV-miHTT in the brain, the expression level of the therapeutic miHTT in the left cortical areas of Q175KI HET was evaluated. Mice were intrastriatally injected with two different doses of AAV-miHTT and vehicle (ns=10 per group) at 5 months of age. Two months after the injection mice were sacrificed, and left frontal and caudal cortex were separately collected. In order to measure the miHTT expression levels in the brain, RT-qPCR was performed. In both frontal and caudal cortex, we detected dose-dependent levels of miHTT after treatment (FIGS. 12A and 12B). In the frontal cortex, there was a higher expression of miHTT, up to 3.2×10³ molecules per cells in mice treated with the high dose (FIG. 12A). In the caudal cortex, we also measured high levels of expression of miHTT, up to 8×10² molecules per cell in mice treated with high dose (FIG. 12B). Based on previous studies, it is expected that the levels of miHTT in the striatum, site of injection, are approx. 10 times higher than in cortex.

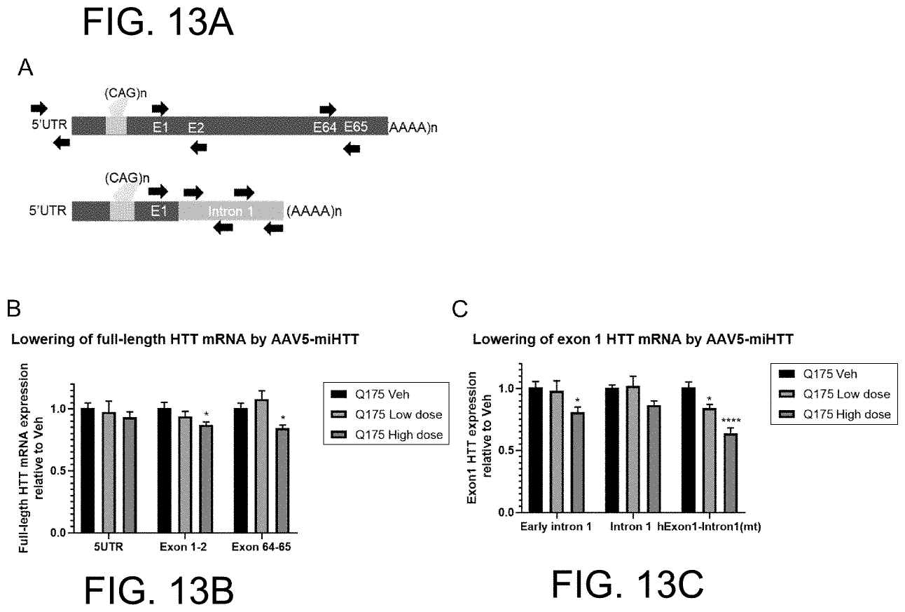

To evaluate the lowering of exon 1 and full-length HTT at the RNA level, the expression level of different HTT sequences was measured by RT-qPCR using different set of primers and probes (FIGS. 9A-9E). Primers were designed to selectively measure the full-length HTT mRNA (primer set “5′UTR”, “exon 1-2” and “exon 64-65”) or the Exon 1 HTT mRNA (“Early intron 1”, “Intron 1” and “human exon1-intron1”) (FIG. 13A). The expression levels in left frontal cortex of AAV-miHTT-treated Q175KI HET mice were compared with vehicle-treated mice to determine the on-target lowering of full-length and Exon1 HTT mRNA in cortical brain areas at 2 months after treatment (n=10 per treatment). In the frontal cortex, distal from injection site, we observed a significant approx. 15% lowering of “exon 1-2” and “exon 64-65” sequence in mice treated with the high dose of AAV-miHTT, but not with the lower dose (FIG. 13B). In the same manner, a significant lowering of the “early intron 1” sequence and mutant “human exon1-intron1” mRNA was detected in the frontal cortex upon treatment with high dose AAV-miHTT, but not with the low dose treatment (FIG. 13C). As previously showed in other studies, it is likely that the levels of miHTT in the cortex after a low dose intrastriatal injection are not sufficient to induce a significant lowering of HTT mRNA. Therefore, it is expected that a higher miHTT biodistribution in striatum, site of injection, results in stronger lowering of exon 1 HTT and full-length HTT mRNA at both doses of injection.

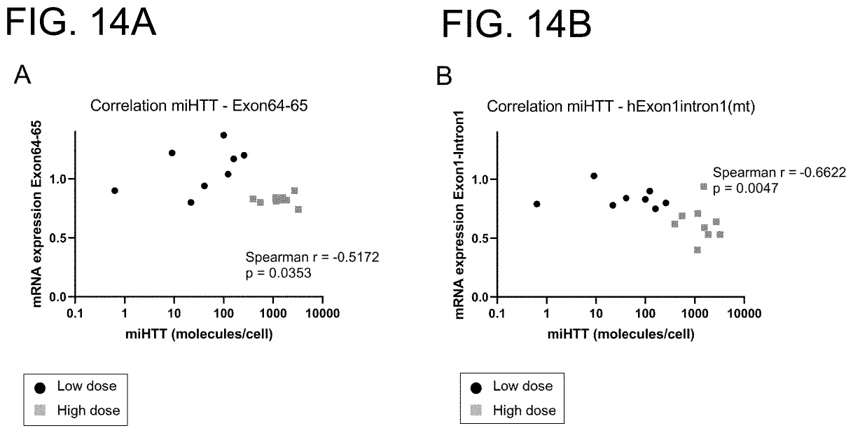

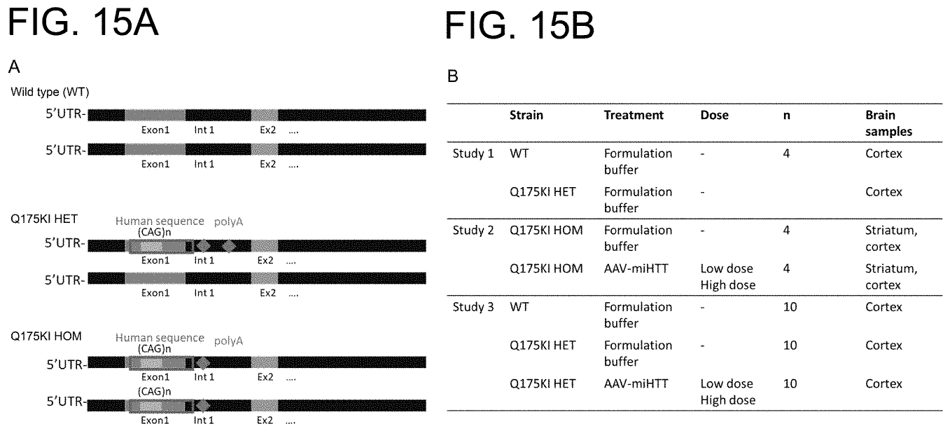

Correlation analysis between miHTT expression levels and HTT mRNA expression showed that there is a significant correlation between miHTT molecules/cell and lowering of full-length HTT mRNA (“exon 64-65”) and between miHTT molecules/cell and lowering of exon 1 HTT mRNA (human exon1-intron1) (FIGS. 14A and 14B).

All together, these results confirmed that AAV-delivered miHTT can target and significantly lower the mis-spliced Exon 1 HTT mRNA in vivo.

The results obtained at the transcript level are confirmed with analyses of HTT protein expression, where region-specific and dose-dependent effects are observed when analyzing different HTT protein species (exon1 HTT, soluble HTT and aggregated HTT), in line with the lowering of HTT transcripts.

1. A method of treatment of a repeat expansion disorder, wherein the repeat expansion is a CAG repeat and results in missplicing 3′ from the repeat expansion, producing a misspliced transcript, the method comprising administering a polynucleotide capable of inducing a reduction of the misspliced transcript.

2. The method according to claim 1, wherein the misspliced transcripts contain (i) an exon comprising the CAG repeat and (ii) an intron sequence, which is 3′ and adjacent from the exon with the CAG repeat.

3. The method according to claim 2, wherein the misspliced transcripts comprise a polyA 3′ adjacent to the intron sequence.

4. The method according to claim 1, wherein the reduction of misspliced transcripts is observed in the cytoplasm.

5. The method according to claim 1, wherein the polynucleotide is complementary to the misspliced transcript.

6. The method according to claim 5, wherein the complementarity is 5′ from the repeat expansion.

7. The method according to claim 1, wherein the polynucleotide is comprised in a double stranded polynucleotide capable of inducing RNA interference.

8. The method according to claim 1, wherein the misspliced transcript encodes a truncated polyQ protein and induces a reduction of the truncated polyQ protein.

9. The method according to claim 2, wherein the repeat expansion disorder is Huntington's Disease, the polynucleotide induces a reduction of misspliced HTT transcripts, the exon with the CAG repeat expansion is exon 1 of HTT, and the intron sequence which is 3′ and adjacent therefrom is from intron 1 of HTT.

10. The method according to claim 9, wherein the polynucleotide is 5′-AAGGACUUGAGGGACUCGAAGA-3′ (SEQ ID NO. 9).

11. The method according to claim 2, wherein the repeat expansion disorder is SCA3, the polynucleotide induces a reduction of misspliced ataxin-3 transcripts, the exon with the CAG repeat expansion is exon 10 of ataxin-3, and the intron sequence which is 3′ and adjacent therefrom is from intron 10 of ataxin-3.

12. The method according to claim 11, wherein the polynucleotide is:
(SEQ ID NO. 22)
5′-UUUCUAACUGUAAACCAGUGUU-3′;
(SEQ ID NO. 23)
5′-UUAAACCACUGUUUUCCUAAUU-3′;
(SEQ ID NO. 24)
5′-UCUGGAACUACCUUGCAUACUU-3′;
(SEQ ID NO. 25)
5′-CUUCCGAAGCUCUUCUGAAGUA-3′;
or
(SEQ ID NO. 26)
5′-UUCAAAGUAGGCUUCUCGUCUC-3′.

13. A method for the treatment of a repeat expansion disorder resulting in missplicing 3′ from the repeat expansion, producing a misspliced transcript, the method comprising administering a polynucleotide capable of inducing a reduction of the misspliced transcript.

14. The method according to claim 13, wherein the reduction of misspliced transcripts is observed in the cytoplasm.

15. The method according to claim 13, wherein the polynucleotide is complementary to the misspliced transcript.

16. The method according to claim 15, wherein the complementarity is 5′ from the repeat expansion.

17. The method according to claim 13, wherein the polynucleotide is comprised in a double stranded polynucleotide capable of inducing RNA interference.

18. The method according to claim 13, wherein the misspliced transcript encodes a truncated polyQ protein and induces a reduction of the truncated polyQ protein.

19. A gene delivery vector encoding a polynucleotide capable of inducing a reduction of the misspliced transcript resulting in missplicing 3′ from a repeat expansion.

20. The vector according to claim 19, which is an AAV gene delivery vector of serotype 5 and the polynucleotide is comprised in a miR451 scaffold.
